(19) United States
(12) Patent Application Publication
(10) Pub. No.: US 2026/0146060 A1
(43) Pub. Date: May 28, 2026

(54) PHOSPHORAMIDITE CONJUGATES AND METHODS OF FORMING SYNTHETIC OLIGONUCLEOTIDES THEREFROM

(71) Applicant: CARNEGIE MELLON UNIVERSITY, PITTSBURGH, PA (US)

(72) Inventors: Krzysztof Matyjaszewski, Pittsburgh, PA (US); Subha Ranjan Das, Pittsburgh, PA (US); Jaepil Jeong, Atlanta, GA (US); Grzegorz Szczepaniak, Warsaw (PL)

(21) Appl. No.: 19/120,947

(22) PCT Filed: Oct. 13, 2023
(86) PCT No.: PCT/US2023/076793

§ 371 Date: Apr. 14, 2025

Related U.S. Application Data
(60) Provisional application No. 63/415,712, filed on Oct. 13, 2022.

(51) Int. Cl. C07H 21/02; C07H 1/00; C07H 21/04

(52) U.S. Cl. CPC C07H 21/02; C07H 1/00; C07H 21/04

(57) ABSTRACT

A method of synthesizing a polynucleotide composition includes performing a synthesis of the polynucleotide composition via phosphoramidite chemistry, solid-phase supported synthesis, to create polynucleotide chain. During at least one cycle of the synthesis, coupling an RDRP-phophoramidite reagent in a growing chain of the polynucleotide composition. The RDRP-phophoramidite reagent, includes a phosphoramidite compound conjugated to an RDRP initiator or a chain transfer agent for RDRP via a moiety comprising a protected hydroxyl group. A hydroxyl protecting group of the protected hydroxyl group is stable to polynucleotide synthesis conditions.

### CROSS-REFERENCE TO RELATED APPLICATIONS

This application claims benefit of U.S. Provisional Patent Application Ser. No. 63/415,712, filed Oct. 13, 2022, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Combining biomacromolecules with synthetic polymers has created new classes of biomaterials that exhibit the properties of both natural and synthetic components. These molecular chimeras often exhibit synergetic and enhanced properties like hyperstability.

Engineering of nucleic acids polymer hybrids (NAPHs) architecture beyond simple diblock copolymers (for example, multiblock copolymers, miktoarm stars and bottlebrush polymers), has been proven to be a fascinating route for tailoring their self-assembly, mechanical and optical properties, pharmacokinetics, and pharmacodynamics. For example, the micro-miscibility of DNA-pNIPAM (DNA-poly(N-Isopropylacrylamide)) conjugates can be controlled by incorporating the pNIPAM chain into different positions in DNA. For example, the use of rod-like pNIPAM-DNA-pNIPAM triblock copolymers and π-shaped branched conjugates allows for the modulation of self-assembly, enabling the formation of various bicontinuous phases beyond lamellar structures. Additionally, the incorporation of PEG (polyethylene glycol) chains or its analogues at both the 5′ and 3′ termini of DNA and RNA or within the middle region has improved the degradation resistance of the nucleic acid to nucleases (exo- or endonuclease) and allowed self-delivery to cells resulting in enhanced therapeutic efficacy.

Until recently, the architecture control has been mainly accomplished by incorporating pre-synthesized polymers into the desired site of the oligonucleotide sequence via coupling reactions such as Cu(I)-catalyzed azide-alkyne cycloaddition (CuAAC), strain-promoted azide-alkyne click chemistry reaction (SPAAC), amide bond formation, base-paring, or enzymatic incorporation. These methods, referred to as the ‘grafting-to’ approach, allow synthesis of block copolymers or simply-branched hybrids. However, access to NAPHs with more complex architectures is limited by steric hindrance and solubility differences between the nucleic acids and synthetic polymers. As a consequence, the control over the grafting density or the number of polymer chains remains challenging despite the specificity of the coupling chemistry. Hence such polymer tethers are often limited to low molecular-weight polymers or oligomers. Moreover, the ‘grafting-to’ approach requires the separation of the unreacted polymers which further impedes the expanding of the field.

A promising alternative is the ‘grafting-from’ method, where polymerization is initiated from the host biomolecule. This approach expands the range of complexity by significantly reducing the steric challenge. Reversible deactivation radical polymerization (RDRP) methods may be utilized for the preparation of modified polynucleotides via a grafting-from approach. The three most common RDRP methods are atom transfer radical polymerization (ATRP), nitroxide mediated polymerization (NMP) and reversible addition fragmentation chain-transfer (RAFT) systems. ATRP and RAFT polymerization are the most widely used polymerization techniques to grow well-defined polymers from, or in the presence of biomolecules, exosomes, and cells, due to their controlled radical polymerization behavior. In ATRP, the polymer chain is grown from an alkyl halide initiator (R—X, X═Cl or Br) through the reversible activation and deactivation processes typically mediated by copper catalysts. In contrast, RAFT polymerization is mediated and controlled by a chain-transfer agent (CTA). Nevertheless, the precise positioning of multiple initiators (for ATRP) or CTAs (for RAFT) within the host nucleic acids and the purification of the final product has been challenging. As a consequence, to date, most reported products have been restricted to the nucleic acid-block copolymers.

A technique for the successful synthesis of precision NAPHs with the desired architecture needs to address the following challenges: (i) direct and site-controlled incorporation(s) of polymer moieties; (ii) facile purification of desired product; and (iii) grafting well-defined polymer with desired dispersity and length.

### SUMMARY

In one aspect, a method of synthesizing a polynucleotide composition includes performing a synthesis of the polynucleotide composition via phosphoramidite chemistry, solid-phase supported synthesis, to create polynucleotide chain. During at least one cycle of the synthesis, coupling an RDRP-phophoramidite reagent in a growing chain of the polynucleotide composition. The RDRP-phophoramidite reagent includes a phosphoramidite compound conjugated to an RDRP initiator or a chain transfer agent for RDRP via a moiety including a protected hydroxyl group. A hydroxyl protecting group of the protected hydroxyl group is stable to polynucleotide synthesis conditions. The hydroxyl protecting group may, for example, be a dimethoxytrityl group or a monomethoxytrityl group. The method may further include, after coupling the RDRP-phophoramidite reagent, removing the hydroxyl protecting group therefrom to form a hydroxyl group, and coupling one of a phosphoramidite nucleoside or another RDRP-phosphoramidite reagent via the hydroxyl group. An RDRP-phophoramidite reagent may be coupled in the growing chain in more than one cycle of the synthesis of the polynucleotide composition. The phosphoramidite nucleoside may, for example, be a phosphoramidite deoxyribonucleoside or a phosphoramidite ribonucleoside. The phosphoramidite nucleoside is a phosphoramidite ribonucleoside in a number of embodiments.

In a number of embodiments, the RDRP-phophoramidite reagent has the formula:

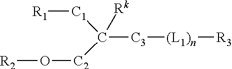

wherein R₁ is a phosphoramidityl group, R₂ is the hydroxyl protecting group, C₁, C₂, and C₃ are independently a base-stable spacer group, R^{k} is selected from the group of H and C1-C6 alkyl, C₃ may be present or absent, L₁ is a base-stable linking group, wherein n is an integer in the range of 0 to 40, and R₃ is the RDRP initiator or the chain transfer agent for RDRP. C₁, C₂, and C₃ may, for example, be independently selected from the group consisting of:

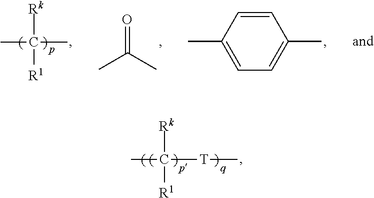

wherein T is selected from the group 0, S, —C(O)NH— or —NHC(O)—, p is an integer in the range of 0 to 18, R^{k} and R^{l} are independently selected from the group consisting of H and C1-C6 alkyl, p′ is an integer in the range of 0 to 18 and q is an integer in the range of 1 to 18.

In a number of embodiments, L₁ is selected from the group consisting of:

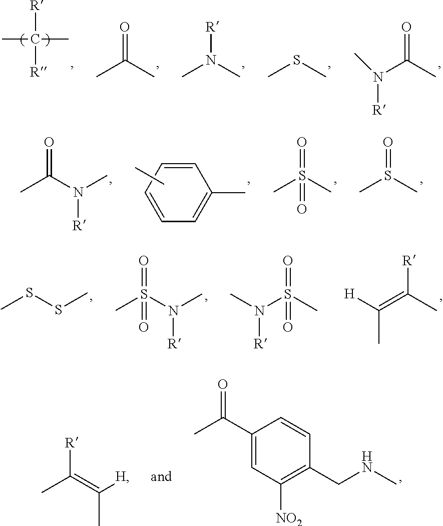

wherein R′ and R″ are independently selected from the group consisting of H, alkyl and aryl.

In a number of embodiments, the phosphoramidityl group has the formula:

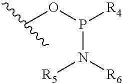

wherein R4 is a phosphate protecting group, and R₅ and R₆ are independently selected from the group consisting of a C1-C12 alkyl group, a C1-C10 branched alkyl group, and a C3-C8 cyclic alkyl group. In a number of embodiments, R₄ is —O—(CH₂)ₙCN, wherein n is an integer in the range of 1 to 5.

In a number of embodiments, R₃ has the formula:

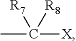

wherein X is a homolytically cleavable group or a group activated by degenerative radical exchange;
R₇ and R₈ are each independently selected from the group consisting of a homolytically cleavable group, a group activated by degenerative radical exchange, H, C1-C20 alkyl, C3-C8 cycloalkyl, C(═Y)R₉, C(═Y)NR₁₀R₁₁, COCl, OH, CN, C2-C20 alkenyl, C2-C20 alkynyl, oxiranyl, glycidyl, aryl, heterocyclyl, aralkyl, aralkenyl, C1-C6 alkyl in which from 1 to all of the hydrogen atoms are replaced with halogen and C1-C6 alkyl substituted with from 1 to 3 substituents selected from the group consisting of C1-C4 alkoxy, aryl, heterocyclyl, C(═Y)R₉, C(═Y)NR₁₀R₁₁, oxiranyl and glycidyl,
wherein R₉ is C1-C20 alkyl, C1-C20 alkoxy, aryloxy or heterocyclyloxy, and R₁₀ and R₁₁ are independently H, or C1-C20 alkyl, or R₁₀ and R₁₁ may be joined together to form an alkylene group of from 2 to 5 carbon atoms, wherein Y is NR₁₂ or O and R₁₂ is H, straight or branched C1-C20 alkyl or aryl.

X may, for example, be selected from the group consisting of Cl, Br, I, nitroxyl, organotellurium, organostibine, organobismuthine, and —S—C(═S)—Z, wherein Z is selected from the group consisting of alkyl, alkoxy, alkylthio, aryl, and heteroaryl. In a number of embodiments, X is selected from the group consisting of Cl, Br, I, —SC(═S)—Z, wherein Z is selected from the group consisting of alkyl, alkoxy, alkylthio, aryl, and heteroaryl. In a number of embodiments, X is selected from the group consisting of nitroxyl, —TeR₁₃, —SbR₁₃R₁₄ and —BiR₁₃R₁₄, wherein R₁₃ and R₁₄ are each independently selected from the group consisting of aryl and a straight or branched C₁-C₂a alkyl group.

In a number of embodiments, R₇, R₈ may, for example, each independently selected from the group consisting of Cl, Br, I, nitroxyl, organotellurium, organostibine, organobismuthine, —S—C(═S)—Z, H, C1-C20 alkyl, C3-C8 cycloalkyl, C(═Y)R₉, C(═Y)NR₁₀R₁₁, COCl, OH, CN, C2-C20 alkenyl, C2-C20 alkynyl, oxiranyl, glycidyl, aryl, heterocyclyl, aralkyl, aralkenyl, C1-C6 alkyl in which from 1 to all of the hydrogen atoms are replaced with halogen and C1-C6 alkyl substituted with from 1 to 3 substituents selected from the group consisting of C1-C4 alkoxy, aryl, heterocyclyl, C(═Y)R₉, C(═Y)NR₁₀R₁₁, oxiranyl and glycidyl. R₇ and R₈ may, for example, each be independently selected from the group consisting of H, C1-C20 alkyl, aryl and a heterocycle. In a number of embodiments, R₇ and R₈ are each independently selected from the group consisting of methyl, phenyl, pyridyl, substituted phenyl, substituted pyridyl and a heterocycle.

In a number of embodiments, C₁ is —CH₂—, and C₂ is —CH₂— or —CHCH₃—, C₂ may, for example, be —CH₂—. In a number of embodiments, C₃ is

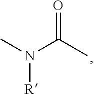

wherein R′ is H and n is 0. In a number of embodiments, X is Br, R₇ is methyl and R₈ is methyl.

The method may further include growing a polymer from the RDRP initiator or from a site of the chain transfer agent for RDRP via an RDRP polymerization. The RDRP initiator may be an ATRP initiator or the chain transfer agent for RDRP is a chain transfer agent for RAFT. The polymer may be grown from the RDRP initiator or from the site of the chain transfer agent for RDRP while the polynucleotide composition is attached to a solid support used in the solid-phase supported synthesis. Alternatively, the polymer may be grown from the RDRP initiator after the polynucleotide chain is detached from a solid support used in the solid-phase supported synthesis. The RDRP initiator is an ATRP initiator and the RDRP is ATRP in a number of embodiments.

In another aspect, a composition include a phosphoramidite compound conjugated to an RDRP initiator or a chain transfer agent for RDRP via a moiety including a protected hydroxyl group, wherein a hydroxyl protecting group of the protected hydroxyl group is stable to polynucleotide synthesis conditions. The hydroxyl protecting group may, for example, be a dimethoxytrityl group or a monomethoxytrityl group.

The composition may have the formula:

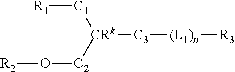

wherein R₁ is a phosphoramidityl group, R₂ is a hydroxyl protecting group, R^{k} is selected from the group consisting of H and C1-C6 alkyl, C₁, C₂, and C₃ are independently a base-stable spacer group, C₃ may be present or absent, L₁ is a base-stable linking group, n is an integer in the range of 0 to 40, and R₃ is the RDRP initiator or the chain transfer agent for RDRP. In a number of embodiments, R₂ is a dimethoxytrityl group or a monomethoxytrityl group.

As described above, C₁, C₂, and C₃ may be independently selected from the group consisting of:

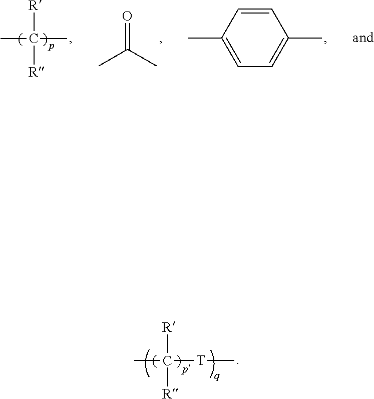

wherein T is selected from the group O, S, —C(O)NH— or —NHC(O)—, R′ and R″ are independently selected from the group consisting of H, alky, and aryl, p is an integer in the range of 0 and 18, p′ is an integer between 0 and 18, and q is an integer in the range of 1 to 18.

As also described above, L₁ may selected from the group consisting of:

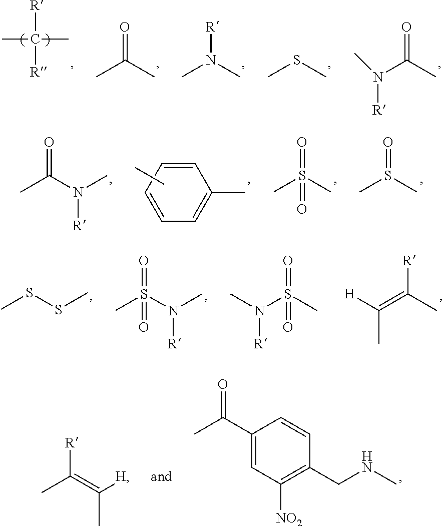

wherein R′ and R″ are independently selected from the group consisting of H, alkyl and aryl.

The phosphoramidityl group may have the formula:

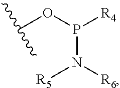

wherein R₄ is a phosphate protecting group, and R₅ and R₆ are independently selected from the group consisting of a C1-C12 alkyl group, a C1-C10 branched alkyl group, and a C3-C8 cyclic alkyl group. In a number of embodiments, R₄ is —O—(CH₂)ₙCN, wherein n is an integer in the range of 1 to 5.

As described above, R₃ may have the formula:

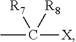

wherein X is a homolytically cleavable group or a group activated by degenerative radical exchange;
R₇ and R₈ are each independently selected from the group consisting of a homolytically cleavable group, a group activated by degenerative radical exchange, H, C1-C20 alkyl, C3-C8 cycloalkyl, C(═Y)R₉, C(═Y)NR₁₀R₁₁, COCl, OH, CN, C2-C20 alkenyl, C2-C20 alkynyl, oxiranyl, glycidyl, aryl, heterocyclyl, aralkyl, aralkenyl, C1-C6 alkyl in which from 1 to all of the hydrogen atoms are replaced with halogen and C1-C6 alkyl substituted with from 1 to 3 substituents selected from the group consisting of C1-C4 alkoxy, aryl, heterocyclyl, C(═Y)R₉, C(═Y)NR₁₀R₁₁, oxiranyl and glycidyl,
wherein R₉ is C1-C20 alkyl, C1-C20 alkoxy, aryloxy or heterocyclyloxy, and R₁₀ and R₁₁ are independently H, or C1-C20 alkyl, or R₁₀ and R₁₁ may be joined together to form an alkylene group of from 2 to 5 carbon atoms, wherein Y is NR₁₂ or O and R₁₂ is H, straight or branched C1-C20 alkyl or aryl. The composition and the substituent groups thereof may further be characterized as described above.

In a further aspect, a polynucleotide composition is formed via a synthesis which is phosphoramidite chemistry, solid-phase supported synthesis, including coupling an RDRP-phophoramidite reagent in a growing chain during at least one cycle of the synthesis. As described above, the RDRP-phophoramidite reagent includes a phosphoramidite compound conjugated to an RDRP initiator or a chain transfer agent for RDRP via a moiety including a protected hydroxyl group. A hydroxyl protecting group of the protected hydroxyl group is stable to polynucleotide synthesis conditions. After coupling the RDRP-phophoramidite reagent, the hydroxyl protecting group is removed to form a hydroxyl group. A phosphoramidite nucleoside or another RDRP-phosphoramidite reagent is then coupled to the growing chain via the hydroxyl group. A polymer is grown from the RDRP initiator or from a site of the chain transfer agent via an RDRP. The polymer may be grown from the RDRP initiator or from the site of the chain transfer agent for RDRP while the polynucleotide chain is attached to a solid-phase support used in the synthesis or after removal therefrom.

In a number of embodiments, the RDRP initiator is an ATRP initiator and the RDRP polymerization is ATRP. The polymer may be grown from the ATRP initiator before or after the polynucleotide chain is detached from a support used in the synthesis.

In still a further aspect, a composition includes a modified polynucleotide chain having at least one repeat unit having the formula:

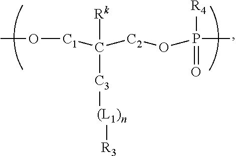

wherein C₁, C₂, and C₃ are independently a base-stable spacer group, C₃ may be present or absent, R^{k} is selected from the group consisting of H and C1-C6 alkyl, L₁ is a base-stable linking group, n is in the range of 0 to 40, and R₃ is an RDRP initiator, a chain transfer agent for RDRP, a polymer grown from the RDRP initiator, or a polymer grown from the chain transfer agent for RDRP, and R₄ is a phosphate protecting group or O or S. C₁, C₂, C₃, R^{k}, L₁, and R₃ may be further characterized as described above. More than one of the repeat unit may be included in the modified polynucleotide chain. Other repeat units in the chain are nucleotide repeat units as occur in DNA. RNA, peptide nucleic acids (PNA), locked nucleic acid (LNA), and hybrids thereof, as well as derivative or analogs thereof.

As used herein the terms “alkyl” (typically, C1-C20, that is, 1 to 20 carbon atoms), “alkenyl” (typically, C2-C20)” and “alkynyl” (typically, C2-C20) refer to straight-chain or branched groups (except for C1 and C2 groups). This definition also application to alkyl groups as a substituent in, for example, alkoxyl. “Alkenyl” and “alkynyl” groups may have sites of unsaturation at any adjacent carbon atom position(s) as long as the carbon atoms remain tetravalent, but − or terminal (i.e., at the − and (−1)-positions) are present in a number of embodiments.

As used herein “aryl” refers to phenyl, naphthyl, phenanthryl, phenalenyl, anthracenyl, triphenylenyl, fluoranthenyl, pyrenyl, pentacenyl, chrysenyl, naphthacenyl, hexaphenyl, picenyl and perylenyl (preferably phenyl and naphthyl), in which each hydrogen atom may be replaced with alkyl of from 1 to 20 carbon atoms (preferably from 1 to 6 carbon atoms and more preferably methyl), alkyl of from 1 to 20 carbon atoms (preferably from 1 to 6 carbon atoms and more preferably methyl) in which each of the hydrogen atoms is independently replaced by a halide (preferably a fluoride or a chloride), alkenyl of from 2 to 20 carbon atoms, alkynyl of from 1 to 20 carbon atoms, alkoxy of from 1 to 6 carbon atoms, alkylthio of from 1 to 6 carbon atoms, C3-C8 cycloalkyl, phenyl, halogen, NH₂, C1-C6-alkylamino, C1-C6-dialkylamino, and phenyl which may be substituted with from 1 to 5 halogen atoms and/or C1-C4 alkyl groups. (This definition of “aryl” also applies to the aryl groups in “aryloxy” and “aralkyl.”) Thus, phenyl may be substituted from 1 to 5 times and naphthyl may be substituted from 1 to 7 times (preferably, any aryl group, if substituted, is substituted from 1 to 3 times) with one of the above substituents. More preferably, “aryl” refers to phenyl, naphthyl, phenyl substituted from 1 to 5 times with fluorine or chlorine, and phenyl substituted from 1 to 3 times with a substituent selected from the group consisting of alkyl of from 1 to 6 carbon atoms, alkoxy of from 1 to 4 carbon atoms and phenyl. Most preferably, “aryl” refers to phenyl and tolyl.

In the context of the present invention, “heterocyclyl” refers to pyridyl, furyl, pyrrolyl, thienyl, imidazolyl, pyrazolyl, pyrazinyl, pyrimidinyl, pyridazinyl, pyranyl, indolyl, isoindolyl, indazolyl, benzofuryl, isobenzofuryl, benzothienyl, isobenzothienyl, chromenyl, xanthenyl, purinyl, pteridinyl, quinolyl, isoquinolyl, phthalazinyl, quinazolinyl, quinoxalinyl, naphthyridinyl, phenoxathiinyl, carbazolyl, cinnolinyl, phenanthridinyl, acridinyl, 1,10-phenanthrolinyl, phenazinyl, phenoxazinyl, phenothiazinyl, oxazolyl, thiazolyl, isoxazolyl, isothiazolyl, and hydrogenated forms thereof known to those in the art. Preferred heterocyclyl groups include pyridyl, furyl, pyrrolyl, thienyl, imidazolyl, pyrazolyl, pyrazinyl, pyrimidinyl, pyridazinyl, pyranyl and indolyl, the most preferred heterocyclyl group being pyridyl. Accordingly, suitable vinyl heterocycles to be used as a monomer in the present invention include 2-vinyl pyridine, 4-vinyl pyridine, 2-vinyl pyrrole, 3-vinyl pyrrole, 2-vinyl oxazole, 4-vinyl oxazole, 5-vinyl oxazole, 2-vinyl thiazole, 4-vinyl thiazole, 5-vinyl thiazole, 2-vinyl imidazole, 4-vinyl imidazole, 3-vinyl pyrazole, 4-vinyl pyrazole, 3-vinyl pyridazine, 4-vinyl pyridazine, 3-vinyl isoxazole, 3-vinyl isothiazoles, 2-vinyl pyrimidine, 4-vinyl pyrimidine, 5-vinyl pyrimidine, and any vinyl pyrazine, the most preferred being 2-vinyl pyridine. The vinyl heterocycles mentioned above may bear one or more (preferably 1 or 2) C1-C6 alkyl or alkoxy groups, cyano groups, ester groups or halogen atoms, either on the vinyl group or the heterocyclyl group, but preferably on the heterocyclyl group. Further, those vinyl heterocycles which, when unsubstituted, contain an N—H group may be protected at that position with a conventional blocking or protecting group, such as a C1-C6 alkyl group, a tris-C1-C6 alkylsilyl group, an acyl group of the formula R₁₃CO (where R₁₃ is alkyl of from 1 to 20 carbon atoms, in which each of the hydrogen atoms may be independently replaced by halide, preferably fluoride or chloride), alkenyl of from 2 to 20 carbon atoms (preferably vinyl), alkynyl of from 2 to 10 carbon atoms (preferably acetylenyl), phenyl which may be substituted with from 1 to 5 halogen atoms or alkyl groups of from 1 to 4 carbon atoms, or aralkyl (aryl-substituted alkyl, in which the aryl group is phenyl or substituted phenyl and the alkyl group is from 1 to 6 carbon atoms), etc. (This definition of “heterocyclyl” also applies to the heterocyclyl groups in “heterocyclyloxy” and “heterocyclic ring.”)

In general, any radically polymerizable alkene can, for example, serve as a monomer for a polymerization reaction hereof. In a number of embodiments, monomers suitable for polymerization in the present method include those of the formula:

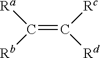

wherein R^{a} and R^{b} are independently selected from the group consisting of H, halogen, CN, straight or branched alkyl of from 1 to 20 carbon atoms (in a number of embodiments, from 1 to 6 carbon atoms, or from 1 to 4 carbon atoms) which may be substituted with from 1 to (2n+1) halogen atoms where n is the number of carbon atoms of the alkyl group (e.g. CF₃), unsaturated straight or branched alkenyl or alkynyl of 2 to 10 carbon atoms (in a number of embodiments, from 2 to 6 carbon atoms, or from 2 to 4 carbon atoms) which may be substituted with from 1 to (2n−1) halogen atoms (in a number of embodiments, chlorine) where n is the number of carbon atoms of the alkyl group (e.g. CH₂═CCl—), C₃-C₈ cycloalkyl which may be substituted with from 1 to (2n−1) halogen atoms (preferably chlorine) where n is the number of carbon atoms of the cycloalkyl group, C(═Y)R^{c}, C(═Y)NR^{f}R^{g}, YC(═Y)R³, SOR^{e}, SO₂₁R³, OSO₂R^{c}, NR^{h}SO₂R^{e}, PR^{e}₂, P(═Y)R^{e}₂, YPR^{e}₂, YP(═Y)R^{e}₂, NR^{h}₂ which may be quaternized with an additional R^{h} group, aryl and heterocyclyl; where Y may be NR^{h} S or O (preferably O); R^{e} is alkyl of from 1 to 20 carbon atoms, alkylthio of from 1 to 20 carbon atoms, ORⁱ (where Rⁱ is H or an alkali metal), alkoxy of from 1 to 20 carbon atoms, aryloxy or heterocyclyloxy; R^{f} and R^{g} are independently H or alkyl of from 1 to 20 carbon atoms, or R^{f} and R^{g} may be joined together to form an alkylene group of from 2 to 7 (preferably 2 to 5) carbon atoms, thus forming a 3- to 8-membered (preferably 3- to 6-membered) ring, and R^{h} is H, straight or branched C₁-C₂₀ alkyl or aryl;
R^{c} and R^{d} are independently selected from the group consisting of H, halogen (preferably fluorine or chlorine), C1-C6 (preferably C₁) alkyl and COOR^{j} (where R^{j} is H, an alkali metal, or a C1-C6 alkyl group), or
R^{a} and R^{b} may be joined to form a group of the formula (CH₂)ₙ, (which may be substituted with from 1 to 2n, halogen atoms or C₁-C₄ alkyl groups) or C(═O)—Y—C(═O), where n′ is from 2 to 6 (in a number of embodiments 3 or 4) and Y is as defined above. In a number of embodiments, at least two of R^{a}, R^{b}, R^{c} and R^{d} are H or halogen.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates synthesis and solid-phase incorporation of SBiB and polymerization from oligonucleotide initiator as follows: (A) Scheme for synthesis of the SBiB phosphoramidite. Abbreviations: Me=methyl group; TEA=triethylamine, EtOH=ethanol; DIPEA=N,N-diisopropylethylamine; NMI=N-methylimidazole; DCM=dichloromethane. (B) Scheme of solid-phase incorporation of SBiB and standard DNA or RNA phosphoramidites using oligonucleotide synthesizer. The inset shows the structure of the incorporated residue. Identical coupling conditions for standard DNA and RNA phosphoramidites were applied for SBiB coupling. Abbreviations: DMT=dimethoxytrityl group; TBDMS=tert-butyldimethylsilyl group. (C) After the solid-phase synthesis, oligonucleotide cleavage and deprotection of phosphate- and base-protecting groups are performed. The DMT group on the desired final product's terminus was utilized as a hydrophobic tag for reverse-phase purification of the oligonucleotide initiator during the standard DMT-on desalting process. Fully deprotected and desalted oligonucleotide initiator was mixed with CuBr₂, TPMA, and EYH₂ for polymer growth via ATRP under green light irradiation in phosphate-buffered saline (PBS).

FIG. 2 illustrates controlled polymerization from the SBiB in the middle of DNA, wherein all the polymerizations were performed under the standard polymerization conditions ([OEOMA₅₀₀]=300 mM, [EYH₂]=0.015 mM, [CuBr₂]=0.9 mM, [TPMA]=2.7 mM) and analyzed by DMF GPC (calibrated to PMMA). T4-SBiB-T4 DNA initiator was used as a model initiator (see Table 4 for the sequence of the DNA initiator); as follows: (A) Schematic illustration of grafting from SBiB located in the center of DNA; inset shows structure of the incorporated SBiB initiator residue. (B) Molecular weight control of the polymer chain grafted from the middle of DNA after 30 minutes of ATRP. The molecular weight was controlled by changing the initiator concentration ([T4-SBiB-T4]=0.3-1.5 mM). (C) First-order kinetic plots of polymerizations using HEBiB (black line) and DNA initiator (red line). (D) GPC traces of the polymer grafted from HEBiB (black line) and DNA (red line) after 60 min of reaction.

FIG. 3 illustrates grafting from DNA with multiple SBiB residue and topology control as follows: (A-C) SEC-MALS traces of DNA with (A) 5 terminal polymer moieties; (B) DNA with five internal polymer moieties; and (C) DNA with 5 spread internal polymers. Standard polymerization conditions at the target DP of 400 were adopted: [DNA initiator]=0.15 mM (i.e., 0.75 mM of SBiB). T8-t-SBiB5, T8-m-SBiB5, and T8-s-SBiB5 were used as the initiator for A, B, and C, respectively. (D) SEC-MALS traces of DNA-polymer hybrids before and after UV cleavage. T12-pc-SBiB3 DNA initiator was used, which contains UV-cleavable linkers between SBiB residue. The polymerization was performed under the standard condition at the target DP of 800: [DNA initiator]=0.125 mM (i.e., 0.375 mM of SBiB). UV light (λ=365 nm, 6.0 mW cm⁻²) was irradiated for 10 min to cleave polymer tethers from DNA. (E) Aqueous GPC (calibrated to poly(acrylic acid) standards) traces of DNA-polymer grafted from 21-mer ssDNA initiator (Beta-SBiB1, black line) and dsDNA initiator (Beta-SBIB1 forming a duplex with Beta*, red line) under the standard polymerization condition (target DP of 600). (F) Schematics of a number of duplexes studied.

FIG. 4 illustrates thermal stability of DNA structures with initiators or polymers. Sequence of the DNA initiators are shown in Table 4. (A) The melting curve analyses of the three types of DNA duplexes: the unmodified DNA duplex (Beta duplex), the DNA duplex with 5 initiators (Beta-SBiB5 duplex), and the DNA duplex with 5 polymers (Beta-p(OEOMA)5 duplex). (B) The melting curve analyses of the three types of DNA hairpins: the unmodified DNA hairpin (Alpha hairpin), the DNA hairpin with 1 initiator in the middle of the loop (Alpha-m-SBiB1), and the DNA hairpin with 1 initiator on the 5′-terminus (Alpha-t-SBiB1). (C) The melting curve analyses of the DNA hairpins with a polymer chain in the middle of the loop (Alpha-m-pOEOMA) or on the 5′-terminus (Alpha-t-pOEOMA). (D) Schematics of DNA structures used for melting curve analysis.

FIG. 5 illustrates degradation profile of modified and unmodified DNA in 10% FBS. Beta-p(OEOMA)3 was modified with three poly(OEOMA₅₀₀)₁₉₄ tethers. The amount of remaining DNA was determined by measuring the intensity of stained DNA after gel electrophoresis. Standard deviations were calculated from three different experiments. pOEOMA (M_{n,MALS}=169 000) initiated from HEBiB was used as the free polymer in the supernatant.

FIG. 6 illustrates controlled polymerization from RNA initiator wherein: (A) The kinetic plots of the logarithm of [M]₀/[M] over polymerization time. 20mer uridine oligo with terminal SBiB (U20-SBiB) was used as a model initiator for the EY/Cu-catalyzed ATRP of OEOMA₅₀₀. Optimized polymerization conditions were employed: [OEOMA₅₀₀]/[I]/[EYH₂]/[CuBr₂]/[TPMA]=600/1/0.03/1.8/5.4, [OEOMA₅₀₀]=300 mM, in PBS under the green light irradiation for 60 min. (B) SEC-MALS trace of RNA-poly(OEOMA₅₀₀) after 60 min of photo-ATRP. (C) Molecular weight control of polymer chains grafted from RNA initiator. U20-SBiB was used as a model initiator for the EY/Cu-catalyzed ATRP of OEOMA₅₀₀. Standard polymerization condition was employed: [OEOMA₅₀₀]]/[I]/[EYH₂]/[CuBr₂]/[TPMA]=600/0.3-1.5/0.03/1.8/5.4, [OEOMA₅₀₀]=300 mM, in PBS under the green light irradiation for 30 min. (D-E) Investigation of simultaneous polymerizations from a single RNA strand. (D) Schematic illustration of grafting from polymerization and subsequent UV irradiation. (E) SEC-MALS traces of RNA-poly(OEOMA₅₀₀) before and after UV irradiation. U12-pc-SBiB2 RNA initiator was used which contains UV-cleavable linkers between SBiB residue. The polymerization was performed under the standard condition at the target DP of 800: [RNA initiator]=0.188 mM (i.e., 0.375 mM of SBiB).

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a needle” includes a plurality of such needles and equivalents thereof known to those skilled in the an, and so forth, and reference to “the needle” is a reference to one or more such needles and equivalents thereof known to those skilled in the art, and so forth. Recitation of ranges of values herein are merely intended to serve as a shorthand method of referring individually to each separate value falling within the range. Unless otherwise indicated herein, each separate value, as well as intermediate ranges, are incorporated into the specification as if individually recited herein. All methods described herein can be performed in any suitable order unless otherwise indicated herein or otherwise clearly contraindicated by the text.

In a number of representative embodiments hereof, strategies for the preparation of modified oligonucleotides or polynucleotides such as polynucleotide-polymer hybrids are set forth. Modified polynucleotides hereof may include one or more incorporated initiators, one or more incorporated chain transfer agents, or one or more incorporated polymerizable moieties or handles. Compositions hereof further include polymers formed from such modified polynucleotides. Examples of suitable polynucleotides include, but are not limited to, polynucleotides and oligonucleotide sequences, including DNA, RNA, peptide nucleic acids (PNA), locked nucleic acid (LNA), hybrids thereof, and derivative or analogs thereof and include, without limitation, synthetic polynucleotides that may be administrated to a patient.

The term “polymer” or the prefix “poly” (when referring to a particular type of polymer such as a polynucleotide) refers generally to a molecule, the structure of which includes repeat units derived, actually or conceptually, from molecules of low relative molecular mass (monomers). The term “oligomer” or the prefix “oligo” (when referring to a particular type of oligomer such as an oligonucleotide) refers generally to a molecule of intermediate relative molecular mass, the structure of which includes a small plurality of units derived, actually or conceptually, from molecules of lower relative molecular mass (monomers). In general, a polymer is a compound having >1, and more typically >13 repeat units or monomer units, while an oligomer is a compound having >1 and <20, and more typically less than 13 repeat units or monomer units. The term polymer thus includes oligomers as well as molecules of higher molecular weight.

To address the challenges set forth above and expand the architectural horizon of nucleic acid-polymer biohybrids, phosphoramidite chemistry, a synthetic method used for oligonucleotide synthesis, was adapted to controllably attach RDRP initiators or chain transfer agent to polynucleotides such as DNA and RNA. Oligonucleotide synthesis via phosphoramidite chemistry is, for example, discussed in Beaucage, S., and Caruthers, M., Deoxynucleoside phosphoramidites—a new class of key intermediates for deoxypolynucleotide synthesis, Tetrahedron Lett. 22, 1859-1862 (1981) and Kosuri, S., and Church, G. M., Large-scale de novo DNA synthesis: technologies and applications. Nat. Methods, 11, 499-507 (2014). As is well known in the art, the phosphoramidite chemistry method includes coupling of phosphoramidite nucleosides on a solid support to build a growing oligonucleotide chain via a cyclic process. In a first step (sometimes referred to as deblocking or detritylation), a protecting group such as dimethoxytrityl group (DMT) is removed from the first, solid-support-linked nucleoside. During functionalization of the solid support, the protecting group prevents polymerization of the nucleoside. In a subsequent coupling step, the free 5′-OH group of the first, solid-support-linked nucleoside reacts with the phosphorus of an added second (phophoramidite) nucleoside, displacing its diisopropylamino group. In a capping step, unreacted 5′-OH groups are acylated to prevent elongation of sequences with deletions (unincorporated residues). Subsequently added phophoramidite nucleosides are likewise protected. In an oxidation step, the unstable phosphite triester is converted to a stable phosphate triester, which allows the next cycle to proceed. After synthesis, the resultant oligonucleotide is cleaved from the solid support. After cleavage, protecting groups are removed from bases and phosphates. Further, the β-cyanoethyl group on the free oxygen of the phosphate groups is removed to convert it from a phosphate triester to a phosphate diester (phosphodiester).

Through the sequential coupling of phosphoramidite building blocks to the oligonucleotide chain on the solid support, phosphoramidite chemistry may be used for synthesizing sequence-defined polynucleotides such as DNA and RNA with functional moieties in the desired location. In a number of studies hereof, a representative, serinol-based a-bromoisobutyryl (SBiB) phosphoramidite was developed that can be used to precisely incorporate single or even multiple RDRP initiators or CTA for RDRP in an oligonucleotide strand at any location(s). The α-bromoisobutyryl ATRP initiator, instead of CTA for RAFT, was chosen for initial studies for functionalization to the phosphoramidite analog due to the potential hydrolysis of typical dithioester-based CTA during the cleavage of the DNA (or RNA) from a solid support. However, the problems associated with hydrolysis may be overcome by performing polymerization from, for example, RAFT CTA or nitroxide moieties in synthesized polynucleotides hereof before the cleavage of the polynucleotides from the solid-support.

Studies hereof demonstrate using automated solid-phase DNA/RNA synthesis with one or multiple SBiB phosphoramidites incorporated into the sequence at a single-nucleotide precision. The methodology hereof provides a facile, robust, and automated strategy for the preparation of, for example, DNA- or even RNA-SBiB initiators, which are precursors for NAPHs. Compared to DNA-polymer hybrids, engineering the architecture of RNA-polymer hybrids has been underexplored, and only a few examples on the grafting from RNA or RNA-triblock copolymer have been reported. Representative studies hereof also demonstrated that SBiB-based phosphoramidite chemistry can be combined with the green-light-induced ATRP with dual catalysis to graft well-defined polymer chains, vastly expanding access to the NAPHs with complex architectures.

To develop a phosphoramidite reagent with an RDRP initiator or chain transfer agent that can be used in subsequent and multiple coupling cycles during solid-phase oligonucleotide synthesis, a starting or linking compound or material in a number of embodiments hereof includes three functional groups. In that regard, the functional groups may include a hydroxyl group for attaching a phosphoramidite (2-cyanoethyl N,N-diisopropyl phosphoramidite) for coupling, a primary amine for attaching the RDRP initiator or chain transfer agent (for example, α-bromoisobutyrate or other ATRP initiator), and a second hydroxyl group, which may be protected with a protecting group such as a dimethoxytrityl (DMT) group, for extending the sequence. Serinol (H₂NCH(CH₂OH)₂) was selected as a representative starting material in a number of studies hereof since it has the three-carbon chain along with all the required functional groups. The three-carbon linker of the SBiB initiator residue represents a minimal addition to an oligonucleotide phosphate backbone, similar to the 3 carbons of a natural residue between phosphates in the backbone of DNA or RNA. Other starting materials or compounds including the functional groups described above may be used in forming a phosphoramidite reagent with an RDRP initiator or chain transfer agent hereof.

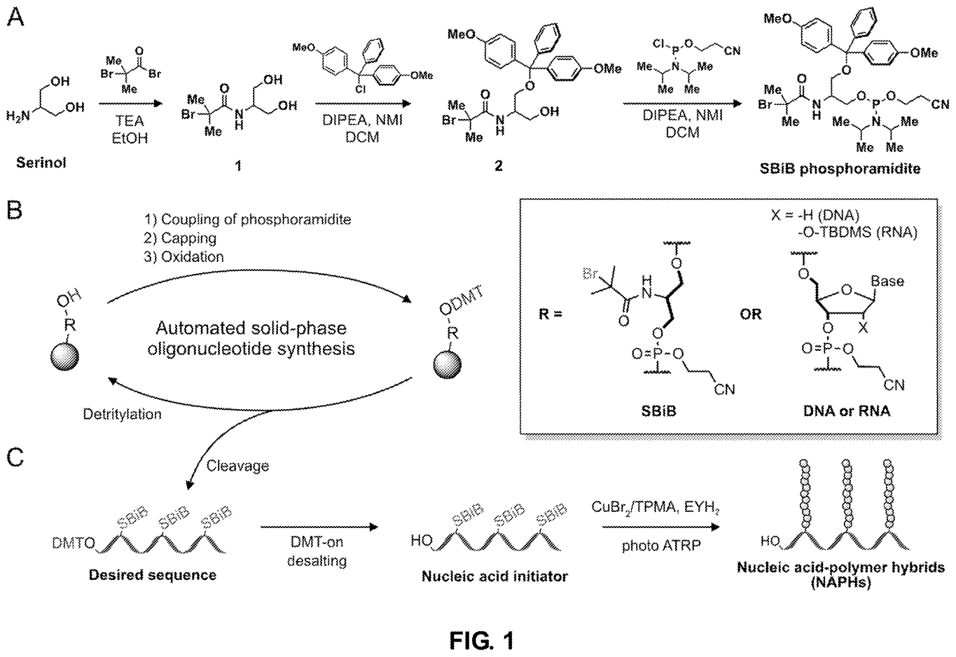

In the synthesis of a representative phosphoramidite, an ATRP initiator such as α-bromoisobutyryl bromide (BiBB) was first attached to the primary amine in the starting material Serinol (FIG. 1A) resulting in the intermediate compound 1. Then one of the hydroxyl groups was protected with a protecting group such as dimethoxytrityl (DMT) group to yield 2. Finally, the reaction with 2-cyanoethyl N,N-diisopropylchlorophosphoramidite yielded the desired SBiB phosphoramidite as a pale-yellow viscous oil. The structure of the final product was confirmed by ¹H NMR and ³¹P NMR.

The SBiB phosphoramidite can be used along with standard DNA, RNA and other phosphoramidites and reagents without modifying the coupling conditions, demonstrating its universality (see Table 1). During solid-phase DNA synthesis in an oligonucleotide synthesizer, the SBiB phosphoramidite was coupled to the 5′-OH on the growing DNA chain (FIG. 1B). In the next step, the DMT group in the incorporated SBiB was removed (for example, with trichloroacetic acid). Then, the deprotected hydroxyl group was used for coupling the next phosphoramidite, which could, for example, either be a standard DNA and RNA phosphoramidite or another SBiB phosphoramidite. Thus, this automated, site-controlled, and direct coupling of SBiB, allowed the incorporation of a polymerization initiator at any desired location within an oligonucleotide sequence, as well as made possible multiple incorporations, for the first time. The same or similar synthetic techniques may be used incorporation of other RDRP initiators or chain transfer agents at any desired location within an oligonucleotide sequence.

After the synthesis, the representative oligonucleotide-SBiB was cleaved from the solid support, deprotected, desalted, and purified. A significant additional benefit of the SBiB phosphoramidite even for terminal attachment is the facile removal of truncated sequences during the desalting step. The final product with the DMT group can be purified by the standard reverse-phase DMT-on desalting process, during which truncated sequences lacking the terminal DMT (having an acetyl group instead of DMT) can be removed during the washing step.

TABLE 1
Polymerization conditions for grafting from DNA macroinitiator and dispersity control.

| Entry | Initiator | [CuBr₂]/[TPMA] | [Cu²⁺] | Conv. | M_{n, th} | M_{n, abs}^{a} | M_{n, GPC}^{b} | Ð^{b} |
|---|---|---|---|---|---|---|---|---|
| 1 | HEBiB | 0.6/1.8 | 0.3 mM | 70% | 210 211 | 230 000 | 149 000 | 1.20 |
| 2 | Free SBiB^{c} | 0.6/1.8 | 0.3 mM | 53% | 159 723 | 182 000 | 123 000 | 1.17 |
| 3 | T4-SBiB-T4 | 0.6/1.8 | 0.3 mM | 80% | 242 970 | 416 000 | 243 000 | 1.35 |
| 4 | T4-SBiB-T4^{d} | 0.6/1.8 | 0.3 mM | 67% | 203 970 | 199 000 | 132 100 | 1.27 |
| 5 | T4-SBiB-T4 | 1.0/3.0 | 0.5 mM | 71% | 215 970 | 317 000 | 194 200 | 1.32 |
| 6 | T4-SBiB-T4 | 1.4/4.2 | 0.7 mM | 67% | 203 970 | 260 000 | 165 100 | 1.29 |
| 7 | T4-SBiB-T4 | 1.8/5.4 | 0.9 mM | 57% | 173 970 | 194 000 | 129 700 | 1.23 |
| 8 | T4-SBiB-T4 | 2.4/7.2 | 1.2 mM | 55% | 167 970 | 122 000 | 88 500 | 1.20 |

Reaction conditions: [OEOMA₅₀₀]/[initiator][EYH₂]/[CuBr₂]/[TPMA] = 600/1/0.03/x/x, [OEOMA₅₀₀] = 300 mM, [initiator] = 0.5 mM, [EYH₂] = 0.015 mM, [CuBr₂] = 0.3-1.2 mM, [TPMA] = 0.9-3.6 mM, in PBS at r.t. irradiated for 30 min under green LEDs (λ = 520 nm, 3.7 mW cm⁻²). GPC traces are shown in FIG. S7. Monomer conversion (Conv.) was determined by ¹H NMR spectroscopy. DNA initiator with an internal SBiB (T4-SBiB-T4) was used as a model DNA oligo initiator.
^{a}Number-average absolute molecular weight (M_{n, abs}) was determined by Mark-Houwink calibration following the previously reported procedures.⁶⁰
^{b}Number-average apparent molecular weight (M_{n, GPC}) and dispersity (Ð) were determined by DMF GPC calibrated to polymethyl methacrylate (PMMA) standards.
^{c}Reaction was conducted in 25% acetonitrile.
^{d}final concentration of NaCl was raised to 340 mM.

With the ability to incorporate the representative SBiB within sequences, the possibility of using the DNA-SBiB as the model macroinitiator for the recently developed green-light-induced ATRP with dual catalysis was next explored. See Szczepaniak, G., Jeong, J., Kapil, K., Dadashi-Silab, S., Yemeni, S. S., Ratajczyk, P., Lathwal, S., Schild, D. J., Das, S. R., and Matyjaszewski, K., Open-air green-light-driven ATRP enabled by dual photoredox/copper catalysis. Chem. Sci. 13, 11540-11550 (2022). That synthetic technique allows controlled aqueous polymerization at a low reaction volume of 250 μL without any deoxygenation process. First, a model oligothymidine that included one SBiB initiator in the middle of a T8 sequence was prepared (T4-SBiB-T4). Oligo(ethylene oxide)methyl ether methacrylate (average Mₙ=500, OEOMA₅₀₀) was used as a model monomer, eosin Y (EYH₂) as the photoredox catalyst, and CuBr₂/TPMA (TPMA=tris(2-pyridylmethyl)amine) as the deactivator. The polymerizations were carried out in phosphate-buffered saline (PBS) solution under the irradiation of green LEDs (λ=520 nm, 3.7 mW cm⁻²) (Table 1). PBS was selected as the reaction medium to ensure benign conditions and convert EYH₂ to its photoactive form (EY). The sequences of all the oligonucleotides used in this study are shown in the Table 4 in the Experimental section.

A number of studies hereof were started by comparing three different initiators 2-hydroxyethyl α-bromoisobutyrate (HEBiB), free SBiB phosphoramidite, and the T4-SBiB-T4 in green-light-induced ATRP, using molar ratios of [OEOMA₅₀₀]/[initiator]/[EYH₂]/[CuBr₂]/[TPMA]=600/1/0.0/0.6/1.8 (Table 1, entries 1-3). All three initiators yielded well-defined polymers with narrow molecular weight distributions (+)<1.35). When the T4-SBiB-T4 initiator was used (Table 1, entry 3), the monomer conversion and the dispersity were higher compared to results using HEBiB (Table 1, entry 1) or free SBiB (Table 1, entry 2) as the initiator. This result may be attributable to the poor halidophilicity of [Cu^{II}/L]²⁺ complexes in aqueous media. The loss of halogen (X) in [X—Cu^{II}/L]⁺ deactivator could cause electrostatic binding of [Cu^{II}/L]²⁺ to the DNA backbone. To test the hypothesis, polymerization from the T4-SBiB-T4 macroinitiator with additional NaCl (final concentration of 340 mM) to suppress dissociation of the [X—Cu/L]²⁺ deactivator and protect DNA phosphate backbone from electrostatic binding of the [Cu^{II}/L]²⁺ complexes was conducted. The monomer conversion decreased from 80% to 67% (Table 1, entry 4), similar to the result with HEBiB (Table 1, entry 1), and a narrower molecular weight distribution was obtained (Ð=1.27). This result indicates that [Cu^{II}/L]²⁺ complexes could interact with DNA predominantly through weak and reversible electrostatic interaction and contamination of nucleic acids with copper through coordination is less preferred due to stable complexation of Cu with ATRP ligands (for example, TPMA). Electrochemical analysis of Cu/TPMA binding to DNA was conducted by progressively adding Salmon DNA to CuBr₂/TPMA complex in PBS. Negligible change in the cyclic voltammetry graph suggested that there is no evident binding of DNA to the catalyst. Further, a series of control experiments, polymerization in the presence of other DNA showed that higher DNA concentrations in the reaction mixture could result in polymers with higher monomer conversion and dispersity values.

The control experiments prompted studies to determine if the dispersity of the DNA-polymer hybrids could be tuned by simply altering the concentration of the Cu complex. As shown in entries 5-8 of Table 1, increasing the CuBr₂/TPMA concentration resulted in lower monomer conversion and dispersity. Under the conditions tested, dispersity values in the range of 1.20 to 1.35 could be achieved. The results indicate that the dispersity of polymer chains grown from DNA can be tuned, offering another route to tailor the properties and functionality of DNA polymer hybrids. Entry 7 in Table 1 was chosen as the standard polymerization conditions and used in subsequent studies hereof because molar ratios of [OEOMA₅₀₀]/[T4-SBiB-T4][EYH₂]/[CuBr₂]/[TPMA]=600/1/0.03/1.8/5.4 provided acceptable monomer conversion (57%) and low dispersity (Ð=1.23) in 30 minutes, along with a reasonable deviation between the theoretical molecular weight (M_{n,th}), the number-average absolute molecular weight (M_{n,abs}) and the apparent molecular weight (M_{n,GPC}).

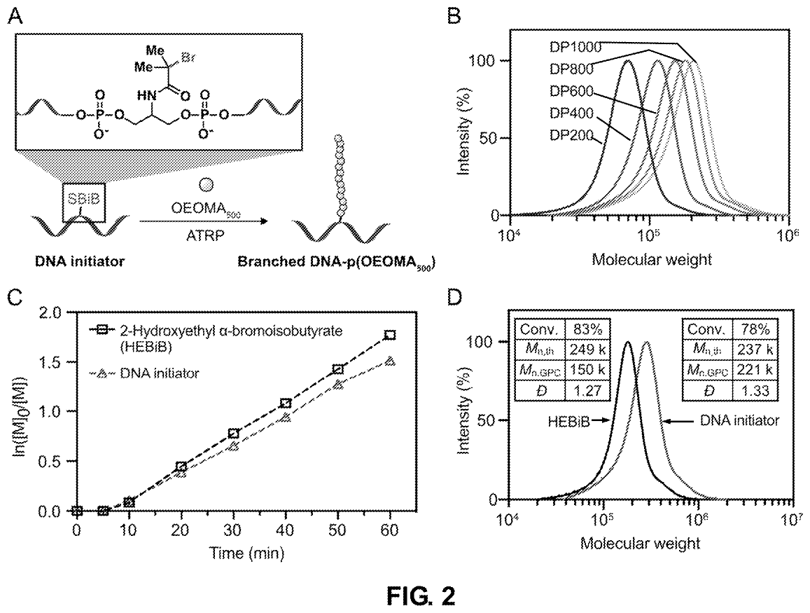

The performance of the T4-SBiB-T4 macroinitiator in EY/Cu-catalyzed ATRP was investigated under optimized conditions (0.9 mM of CuBr₂ and 2.7 mM of TPMA, FIG. 2A through 2D). As demonstrated in FIG. 2B, the DNA initiator with SBiB in the middle showed good molecular weight control, a monomodal GPC trace with low dispersity (Table 2), and followed a behavior of controlled polymerization similar to the HEBiB initiator (FIG. 2C), indicating that there is a little steric constraint at the initiation site. In the studies of Table 2, polymerization reactions were performed under standard polymerization conditions. DMF GPC (calibrated to PMMA) was used to determine Mₙ (number average molecular weight) and Ð (dispersity). Monomer conversion was determined by ¹H NMR spectroscopy. OEOMA₅₀₀ was used as a model monomer. The kinetic study revealed that after a short inhibition period of 5 minutes, the monomer conversion evolved linearly, reaching 78% after 60 minutes of green light irradiation. The resulting DNA-polymer hybrid was further analyzed by GPC, and the result showed a reasonably narrow molecular weight distribution of 1.33 (FIG. 2D). However, the M_{n,GPC} of DNA-polymer conjugates was higher than the result from the control experiment acquired using conventional ATRP initiator (that is., HEBiB). That result may, for example, be due to the termination reaction which can be observed from the decreased monomer consumption between 50-460 min in FIG. 2C.

TABLE 2
GPC traces from FIG. 2B.

| | [T4-SBiB- | | | [M]/[I]/[CuBr₂]/ | | | | |
|---|---|---|---|---|---|---|---|---|
| Entry | T4] | | Target DP | [TPMA]/[EYH₂] | Conv. | M_{n, th} | M_{n, GPC} | Ð |
| 1 | 1.5 | mM | 200 | 200/1/0.6/1.8/0.01 | 74% | 76 700 | 60 620 | 1.18 |
| 2 | 0.75 | mM | 400 | 400/1/1.2/3.6/0.02 | 64% | 130 700 | 95 140 | 1.18 |
| 3 | 0.5 | mM | 600 | 600/1/1.8/5.4/0.03 | 61% | 185 700 | 122 100 | 1.23 |
| 4 | 0.375 | mM | 800 | 800/1/2.4/7.2/0.04 | 57% | 230 000 | 139 200 | 1.26 |
| 5 | 0.3 | mM | 1000 | 1000/1/3.0/9.0/0.05 | 45% | 227 700 | 155 200 | 1.27 |

The steric hindrance to polymerization from an initiator incorporated in the middle of a DNA sequence was also investigated. Three different DNA initiators with different degrees of crowding were prepared (DNA with internal SBiB; DNA with a terminal SBiB; and DNA with a previously reported terminal ATRP initiator that has a four carbon spacer). U.S. Pat. No. 9,765,169 and Averick, S. E., Dey, S. K., Grahacharya, D., Matyjaszewski, K., and Das, S. R., Solid-Phase Incorporation of an ATRP Initiator for Polymer-DNA Biohybrids. Angew. Chem., Int. Ed. 53, 2739-2744(2014), the disclosures of which are incorporated herein by reference. Polymerizations from all the DNA initiators showed compatible conversions (60-64%) and low dispersity (1.18<Ð<1.2). These results demonstrate that the steric hindrance to grafting from the middle of DNA is negligible and that well-controlled polymerization from SBiB within DNA can be expected, regardless of its position, despite the relatively small inter-nucleotide distance (ca., 0.34 nm) compared to commonly used PEG-like monomers (e.g., OEOMA₅₀₀ and OEOMA₃₀₀).

DNA initiators still attached to the solid-support could be useful for the polymerization of hydrophobic monomers, beyond hydrophilic, PEG-like monomers, and the synthesis of nucleic acid-polymer amphiphiles. A number of studies hereof, thus took advantage of the T8-SBiB model DNA initiator still on the CPG solid support and used methyl acrylate (MA) as the model hydrophobic monomer for polymerization. Notably, ethyl α-bromoisobutyrate (EBiB) was added as the sacrificial initiator to increase the deactivator concentration [X—Cu^{II}/L]²⁺ and suppress undesired terminations. After polymerization in DMSO under green light irradiation and subsequent washing following the cleavage process, the resulting DNA-poly(methyl acrylate) block copolymer (DNA-pMA) was characterized by DMF GPC. The small deviation between the theoretical molecular weight and M_{n,GPC}, as well as a low dispersity of 1.15 indicated the successful synthesis of DNA amphiphile by ATRP from the solid support.

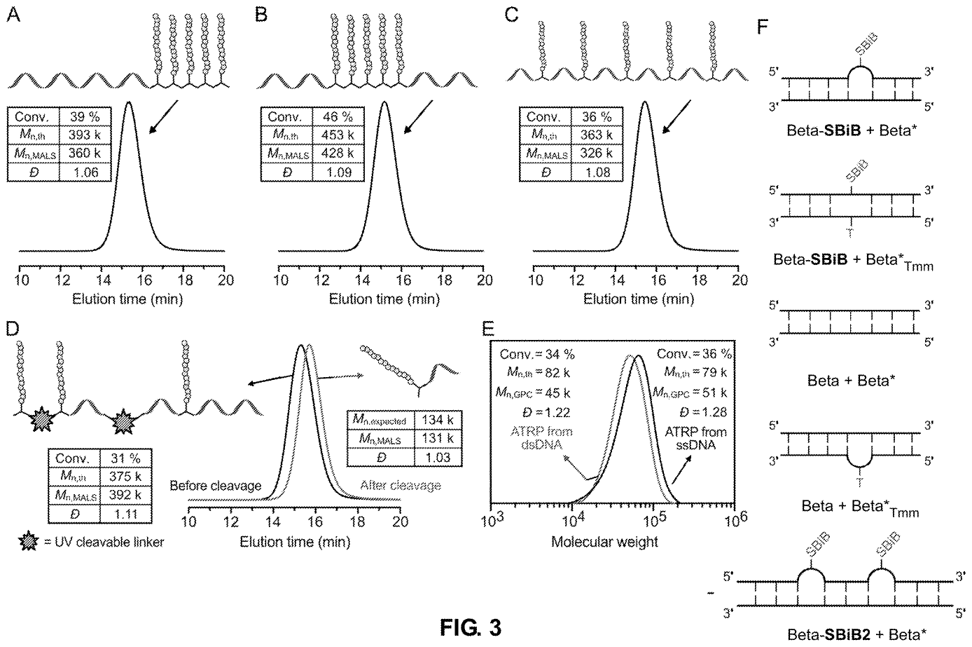

The applicability of the methodology hereof to prepare more complex DNA-polymer hybrids was then studied by incorporating multiple SBiB initiators in different positions in DNA sequences. Three different DNA-SBiB initiators with five SBiB moieties in different positions were prepared: one with five consecutive SBiB initiators, all at the 5′-terminus (T8-t-SBiB5); one with five consecutive SBiBs in the middle of a T8 sequence (T8-m-SBiB5); and five SBiBs spread within a T8 sequence such that there was one thymidine residue between the SBiB units (T8-s-SBiB5). After the polymerization from the DNA initiators, the DNA-polymer hybrid was purified using a 100 k molecular weight cut-off (MWCO) filter and C18 cartridge and characterized by size-exclusion chromatography equipped with multi-angle light scattering detector (SEC-MALS) using PBS as an eluent. The results shown in FIGS. 3A-3C demonstrate that a well-defined polymer chain was grown from each of the DNA initiators. Anly a small deviation between the theoretical molecular weight and M_{n,MALS}, as well as a low dispersity despite the presence of multiple initiators was observe. The results indicate that the method hereof can be used to engineer complex multi-grafted polymer architectures from initiators incorporated with single nucleotide precision within sequences.

For the synthesis of multi-branched bioconjugates, simultaneous initiation is often an important parameter that leads to well-defined concurrent polymer growth from all initiation sites. To more carefully examine the homogeneity of polymers grafted from different SBiB initiators within a single DNA molecule, a T12 DNA initiator with three SBiB residues containing photocleavable (pc) o-nitrobenzyl linkers between SBiB units was designed (T12-pc-SBiB3; FIG. 3D). Initially, photo-ATRP was performed using T12-pc-SBiB3 as the initiator under the optimized polymerization conditions with a target degree of polymerization (DP) of 800. The resulting DNA-polymer hybrid was purified using a 100 k MWCO filter and C18 reverse-phase column to remove unreacted monomer and photocatalyst. The monomodal GPC trace and low dispersity of 1.11 indicated successful polymerization (FIG. 3D). Following that analysis, a vial containing the DNA-polymer hybrid was irradiated with UV light (λ=365 nm, 6.0 mW cm⁻²) for 5 minutes to cleave the photocleavable linker in the DNA backbone and release the polymer chains. These cleaved polymer chains were analyzed by SEC-MALS (FIG. 3D). The released polymers showed a monomodal and symmetrical curve with narrow molecular weight distribution (Ð=1.03). In addition, the number-average molecular weight obtained from SEC-MALS (M_{n,MALS}=131 000) was nearly one-third of the molecular weight obtained before cleavage (M_{n,th}=134 000)—correspondingly well with the 3 different initiators that were on the T12-pc-SBiB3 sequence. It was thereby confirmed that the homogeneity of the multiple polymer chains simultaneously initiated and grafted-from from the SBiB residues integrated into the distinct locations interspersed within a single oligonucleotide molecule.

Hybridization-driven self-assembly has made DNA a versatile building material for nanoscience. The sequence control over SBiB incorporation in DNA can expand the availability of DNA-initiators as new precursors for the fabrication of hybrid nanomaterials. Towards such an end, the hybridization of a mixed oligonucleotide sequence containing an internal SBiB initiator residue in the middle (Beta-SBiB1) was studied with a stoichiometric amount of complementary DNA followed by temperature annealing. For hybridization experiments, two different complementary strands to Beta-SBiB1 were used: one which was fully complementary to the Beta sequence (Beta*) and another that was fully complementary to the Beta sequence with the additional inclusion of a mismatched T residue opposite the SBiB location within the Beta sequence (Beta*_{Tmm}). See FIG. 3F. Beta*_{Tmm} was used to evaluate if the single T residue opposite the 3 carbons insert of SBiB could reduce the strain during duplex formation. Through native gel electrophoresis, it was observed that Beta-SBiB1 could successfully hybridize with both Beta* and Beta*_{Tmm}. Further, it was found that even DNA with two internal SB moieties (Beta-SBiB2) also could form a duplex without leaving unhybridized single-stranded DNA.

To illustrate the potential of SBiB-modified DNA duplexes for nanofabrication, studies were conducted to graft from SBIB in the middle of annealed duplex DNA. Under the duplex DNA initiator, the strands had undergone heating to 95° C. for the annealing process. The general EY/Cu-catalyzed ATRP conditions in 1×PBS at room temperature were used with the DNA duplex initiator, and the polymer hybrid was analyzed by GPC. The polymerization from single-stranded (Beta-SBiB1) and double-stranded DNA (Beta-SBiB1+Beta* duplex) showed comparable conversion and dispersity as shown in FIG. 3E. The conversions from both experiments were slightly lower compared to previous results with shorter oligoT initiator (T4-SBiB-T4, Table 1, entry 7), which could be attributed to the intrinsic nature of grafting from a macroinitiator. Interestingly, the polymer chains initiated from double-stranded DNA (FIG. 3E) showed a slightly narrower molecular weight distribution, which may be due to the bulged-out SBiB from the duplex and the conformational rigidity (persistence length of 50 nm).

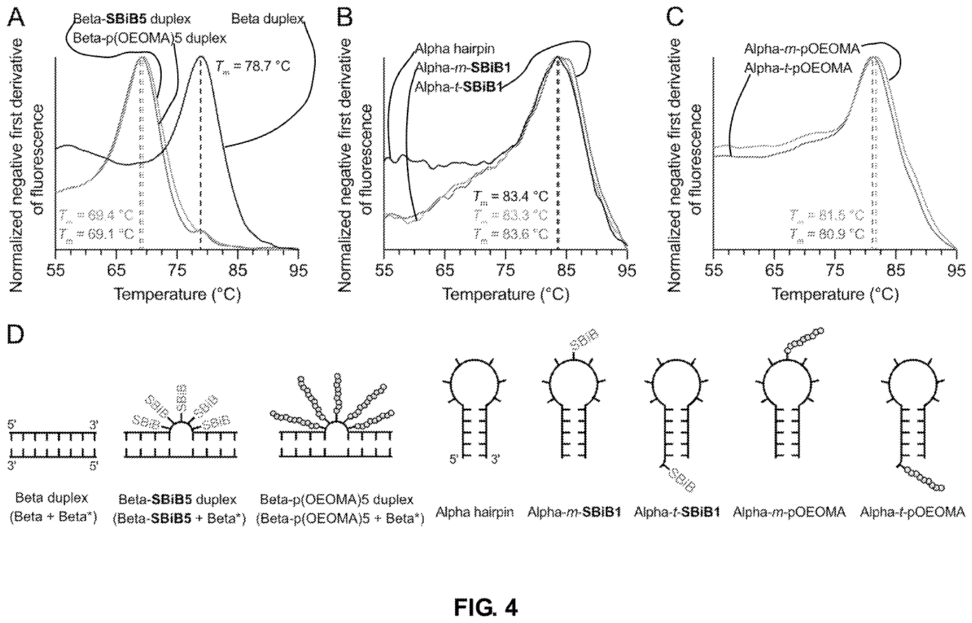

The thermal stability of the Beta-SBiB1/Beta* duplex was then investigated by melting curve analysis. The Beta-SBiB1/Beta* duplex was stained with EvaGreen dye, and the mixture was gradually heated from room temperature to 95° C. while recording the fluorescence drop of EvaGreen using real-time PCR in IX PBS. A slight decrease in the melting temperature (m) of the DNA initiator duplex (68.4° C.) and the DNA-pOEOMA duplex (65.2° C.) was observed compared to the unmodified DNA duplex (73.5° C.), which is similar to that of an abasic residue. The melting temperature of more complex DNA-polymer conjugates was then measured (FIG. 4). For these analyses, a DNA strand containing 5 SBiB residues (Beta-SBiB5) was synthesized and utilized as the initiator yielding the single-stranded DNA with 5 polymer tethers (Beta-p(OEOMA)5, M_{n,MALS}=484 000). The Beta-p(OEOMA)5 was then hybridized with its complementary DNA (Beta*) to form the Beta-p(OEOMA)5 duplex and utilized for melting analysis. 20 mM MgCl₂ was additionally added to the buffer for melting curve analysis to enhance the stability of duplex. The thermal stability of the 21-bp DNA duplex was not significantly affected by the incorporation of 5 polymer chains and a Tₘ of 69.1° C. was observed, which is similar to the DNA initiator duplex (that is, Beta-SBiB5 duplex, Tₘ of 69.4° C.), indicating that the polymer chains are not more disruptive to hybridization than the SBiB inserts in the backbone. This may be attributed to the stronger ionic strength compared to the conditions of melt curve analysis studies as well as the macromolecular excluded volume effect which promotes hybridization. It was further explored whether modifications outside the duplex region affects the hybridization. For this study, DNA hairpins were synthesized with an SBIB initiator positioned at one of two non-duplex locations: either in the middle of the loop (Alpha-m-SBiB1) or extended at the 5′ terminus (Alpha-t-SBiB1). As shown in FIG. 4B, the three hairpin analogues exhibited nearly identical Tₘs for the 12-bp stem. In addition, the data indicates that the polymerization from SBIB in the hairpin initiator did not significantly affect the stability of the duplex. Thus, the hybridization of nucleic acid initiators or their corresponding polymer hybrids can be engineered by strategically positioning the modifications, in addition to ionic strength and sequence designs including sequence length or GC content.

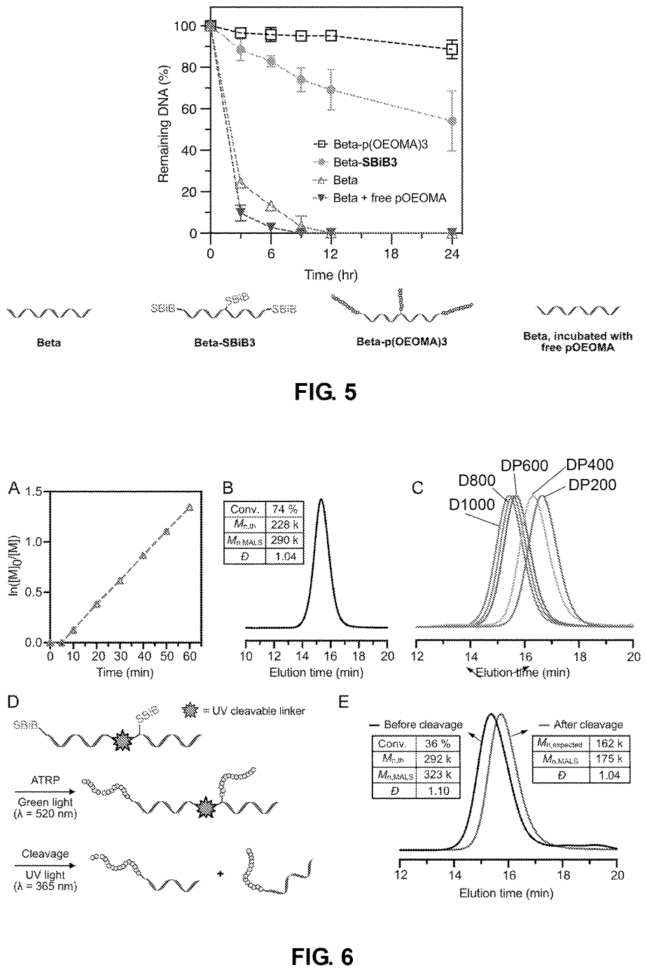

Although the sequence specificity, programmable self-assembly, and bioactivity of nucleic acids make them a critical and promising component of next-generation diagnostics, therapeutics, and biomaterials, the disadvantages of oligonucleotides alone, such as poor bioavailability, cellular uptake, and low nuclease resistance significantly hinder their effectiveness. Chemically modified nucleotides or incorporation of non-natural residues and linkages have been crucial in restricting the enzymatic degradation of nucleic acids. Notably, the size, density, and location of the modified residue affect the stability of DNA. The polymer-assisted stabilization of DNA in 10% fetal bovine serum (FBS) was studied. Beta-SBiB3 and Beta-SBiB1 (middle only) were used as the initiator for polymerization under the optimized conditions, and the resulting Beta-p(OEOMA)3 was purified by MWCO filter and polyacrylamide gel electrophoresis (PAGE). SEC-MALS characterization showed a low dispersity of 1.10 and a molecular weight of 297 000, indicating that the poly(OEOMA)₁₉₃; chains were attached on the 5′, 3′ and in the middle of the Beta sequence. To evaluate their resistance to degradation, unmodified Beta DNA, Beta-SBiB3 initiator, and OEOMA₅₀₀-grafted Beta-SBiB (Beta-p(OEOMA)3) at a final concentration of 5.3 μM in 10% FBS and IX PBS were incubated at 37° C. for 0-24 hours and analyzed by gel electrophoresis. As shown in FIG. 5, unmodified Beta DNA incubated in the presence (filled, inverted triangles) or in the absence (unfilled triangles) of the free pOEOMA in the supernatant were entirely degraded within 12 hours, while 46% degradation of small molecule-modified DNA (Beta-SBiB3, filled circles) after 24 hours was observed. On the other hand, 89% of the Beta-p(OEOMA)3 (unfilled squares) survived after 24 hours of incubation in 10% FBS, mainly because the bulky polymer tether effectively inhibited the binding of nucleases to DNA. The degradation of Beta-p(OEOMA)3 under the treatment of specific nucleases was also investigated. 52% and 7% of Beta-p(OEOMA)3 survived after the treatment of DNase I and Exonuclease VII, respectively, indicating the possible degradation of polymer-modified DNA by both exo- and endonuclease.

The representative SBiB incorporation methodology hereof was also extended to RNA sequences. The solid-phase synthesis of RNA is similar to that of DNA with an important additional step for the final deprotection of the 2-hydroxyl after cleavage from solid-support and deprotection of the nucleobase and phosphate protecting groups. Initially SBiB stability was examined under 2′-hydroxyl deprotection conditions using triethylamine trihydrofluoride (TEA·3HF). Model DNA initiators (T10-SBiB) with or without treatment of TEA·3HF (2.5 hours at 65° C.) were prepared and utilized to benchmark polymerization. The RNA-polymer hybrids were characterized by ¹H NMR and SEC-MALS. The compatible monomer conversion and the M_{n,MALS} was observed regardless of the TEA·3HF treatment, demonstrating the stability of SBiB under 2′-hydroxyl deprotection conditions.

Having demonstrated the additional stability of SBiB to the RNA deprotection conditions the capability of the SBiB residue in RNA to initiate controlled polymerization was studied. First, a 20mer model RNA with SBiB on the 5′-terminus (U20-SBiB) was synthesized using an oligonucleotide synthesizer (MALDI-TOF), and then used for the EY/Cu-catalyzed ATRP of OEOMA₅₀₀ under optimized conditions for DNA hybrids. As shown in the kinetic study of FIG. 6A, the linear growth of ln([M]₀/[M]) over time indicated that the controlled polymerization was carried out from RNA, reaching a high monomer conversion of 74%. After 60 min of polymerization in PBS under green light, the RNA-polymer hybrid was analyzed by SEC-MALS. A low dispersity of 1.04 evidenced the well-controlled polymerization from the RNA initiator. In addition, SEC-MALS traces in FIG. 6C demonstrate that by simply changing the RNA initiator concentration for polymerization, the molecular weight of the grafted polymer can be controlled with a narrow molecular weight distribution (1.02<Ð<1.04).

Simultaneous initiation from RNA initiator by using a U12 RNA initiator with two SBiB residues and a photocleavable linker between SBiB units was also investigated (U12-pc-SBiB2: FIGS. 6D and 6E). Monomodal and symmetrical traces were observed before and after cleavage. In addition, the M_{n,MALS} of the cleaved polymer (FIG. 6E) was nearly half of the molecular weight of the RNA-polymer conjugate before cleavage, indicating fast and concurrent initiation of polymerization from RNA. These results show that the use of the SBiB, even with RNA, remains a versatile and robust method which significantly expands access to possible NAPHs.

The compositions and methodologies hereof provide for the precision fabrication of NAPHs, which have been previously inaccessible. The representative SBiB phosphoramidite hereof, a serinol-based ATRP initiator-modified phosphoramidite can be precisely located in any DNA or RNA sequence during solid-phase synthesis, in single or multiple incorporations. EY/Cu-catalyzed ATRP using the representative SBIB initiator in the middle of an oligonucleotide sequence followed controlled radical behavior with good control over molecular weight and relatively low dispersity (1.18<Ð<1.35). Grafting from a sequence with multiple initiators and UV-induced cleavage of polymer chains confirmed the homogeneity of the polymer tethers grown from a DNA or RNA strand. As set forth above, the three-carbon linker of the SBiB initiator residue represents a minimal addition to an oligonucleotide phosphate backbone, similar to the 3 carbons of a natural residue between phosphates in the backbone of DNA or RNA. Duplex DNA in which the SBiB ‘residue’ may be bulged out could be used to generate a duplex DNA polymer hybrid. It was also demonstrated that the enhanced remarkable degradation resistance of polymer-modified DNA (survival rate of 89%) in 10% FBS. Together these results indicate that the potential application of SBiB in more complex structured DNA-polymer architectures. The studied hereof also demonstrated the first examples of a phosphoramidite approach for incorporating one or more initiators in RNA and subsequent RNA-polymer hybrids.

In combination with the developed representative SBiB phosphoramidite that can be used for incorporation in polynucleotides including both DNA and RNA, oxygen tolerant methods for ATRP use low copper and ligands concentrations that are more effective with DNA and RNA (for example, green-light-induced ATRP with dual catalysis). The method hereof provide opportunities for the expanding the architectural scope of NAPHs in addition to the variety of methods for functionalization of polymers on a phosphate backbone using a diverse library of phosphoramidite reagents—from fluorescent dyes and biofunctional molecules such as biotin and cholesterol to photocleavable linkers that are available for solid-phase synthesis. The methods hereof may also provide the ability to incorporate polymers anywhere in, for example, a folded functional DNA as in quadruplexes, aptamers and DNAzymes, as well as in the abundance of biofunctional folded RNA sequences would provide unprecedented access to new NAPHs for variety of biomedical applications, including drug delivery and biosensing, and microarray.

### EXPERIMENTAL

Materials. 2-Hydroxyethyl α-bromoisobutyrate (HEBiB), α-bromoisobutyryl bromide (BiBB), triethylamine (TEA), diisopropylethylamine (DIPEA), 1-methylimidazole (NMI), methylamine, CuBr₂, eosin Y (EYH₂), Amicon ultra centrifugal filter, and GelGreen dye were purchased from Sigma Aldrich. Poly(ethylene glycol) methyl ether methacrylate monomer (OEOMA₅₀₀, Sigma Aldrich) was passed through a column packed with basic alumina (Fisher Scientific) prior to use. All the organic solvents, 40% PAGE gel mix, ammonium hydroxide, 10× phosphate-buffered saline (PBS), and 10× tris-borate-EDTA buffer were purchased from Fisher Scientific. Tris(2-pyridylmethyl)amine (TPMA) was purchased from AmBeed. N,N,N′,N″,N″-tris[2-(dimethylamino)ethyl]amine (Me₆TREN, 99%) was received from Koei Chemical Co., Ltd. 4-Amino-1-butanol was purchased from TC America. Serinol and dimethoxytrityl chloride (DMT-Cl) were purchased from Combi-Blocks. Unmodified DNA oligos (Beta, Beta*, Beta*_{Tmm}, Alpha hairpin) were purchased from Integrated DNA Technologies (IDT). 1 ml glass shell vial for the kinetic study was purchased from Kimble Chase. 0.5-dram glass vial was purchased from VWR. 250 μL glass inserts for 2 ml vials were purchased from Restek. 12 VDC green LED strip light was purchased from aspectLED. Sep-Pak C18 column was purchased from Waters. Reagents for solid-phase DNA synthesis, PC linker phosphoramidite, and DNA and RNA desalting columns were purchased from Glen Research. Standard DNA phosphoramidites, CPG beads, and 2-cyanoethyl-N,N-diisopropyl-chloro-phosphoramidite (PCI) were purchased from Chemgenes. Columns for DNA synthesis were purchased from Biosearch Technologies. EvaGreen dye was purchased from Biotium. Fetal bovine serum (FBS) was purchased from Gibco, ThermoFisher Scientific. DNAse I and Exonuclease VII were purchased from New England BioLabs.

Instruments. Bruker Avance III 500 MHz spectrometer was used for ¹H nuclear magnetic resonance (¹H NMR) measurements. QuantStudio 3 Real-Time PCR system (ThermoFisher Scientific) was used for melting curve analysis. Mermade 4 (Bioautomation) was used for solid-phase DNA synthesis. NanoDrop One UV-Vis spectrophotometer was purchased from ThermoFisher Scientific. Typhoon FLA 9000 gel scanner (GE Healthcare Life Sciences) was used for fluorescent image scanning after gel electrophoresis. UltrafleXtreme MALDI-TOF Mass Spectrometer (Bruker) was used for the characterization of RNA after synthesis along with MTP 384 Target Plate Ground Steel (Bruker). 3-Hydroxypicolinic acid dissolved in 50% acetonitrile in water containing 10 mg/mL diammonium hydrogen citrate was used as a matrix for MALDI-TOF analysis.

For DMF GPC analysis, Waters 515 HPLC pump equipped with Waters 2414 refractive index detector, and PSS GRAM columns were used in DMF as a running buffer at a flow rate of 1 mjl/min at 50° C. The GPC was calibrated to polymethyl methacrylate (PMMA) standards to achieve apparent molecular weights (M_{n,GPC}) and dispersity (M_{w}/Mₙ).

For aqueous GPC analysis, Waters Alliance 2695 separation module, Waters 2414 refractive index detector using PSS SUPREMA analytical columns were used in PBS as a running buffer at a flow rate of 1 ml/min at 30° C. The GPC was calibrated to poly(ethylene oxide) (PEO) standards to achieve apparent molecular weights (M_{n,GPC}) and dispersity (M_{w}/Mₙ).

Absolute molecular weight (M_{n,MALS}) and dispersity (M_{w}/Mₙ) were determined by Agilent 1260 Infinity ii module with UV detector, multi-angle light scattering detector (DAWN, Wyatt), viscometer (ViscoStar, Wyatt), refractive index detector (Optilab, Wyatt), and Waters Ultrahydrogel linear column using 0.1 M Dulbecco's phosphate buffer saline (DPBS) as a running buffer at a flow rate of 0.5 ml/min at 30° C.

Synthesis of Intermediate Compound 1. Serinol (4.5 g, 50 mmol), triethylamine (5 g, 50 mmol), and KHCO₃ (10 g, 90 mmol) were dissolved in 100 ml ethanol followed by dropwise addition of α-bromoisobutyryl bromide (13.4 g, 60 mmol) in 0-20° C. The reaction was stirred for 16 hours. Then, the mixture was filtered, and the solvent was evaporated. The product was redissolved in 50 ml dichloromethane and precipitated with Hexane. The white precipitates were collected by filtration (yield 80%).

Synthesis of Intermediate Compound 2. Compound 1 (0.95 g, 4 mmol), DIPEA (2.5 g, 20 mmol), and NMI (0.2 g, 2.6 mmol) were dissolved in 20 ml DCM anhydrous, and DMT-Cl (1.7 mg, 5.2 mmol) dissolved in 8 ml DCM was added dropwise in 0-20° C. After 2 hours of reaction with stirring, the mixture was purified by column chromatography (EtOAc/Hexane/TEA, 50:50:0.5) with 40% yield.

Synthesis of SBiB phosphoramidite. Compound 2 (0.46 g, 0.85 mmol), DIPEA (0.55 g, 4.3 mmol), and NMI (0.035 g, 0.43 mmol) were dissolved in 10 ml DCM anhydrous followed by dropwise addition of PCI (0.3 mg, 1.275 mmol) in 0-20° C. After 2 hours of reaction with stirring, the mixture was purified by column chromatography (EtOAc/Hexane/TEA, 50:50:0.25) with 66% yield. The SBiB phosphoramidite was kept in the freezer (−20° C.) until use.

Preparation of DNA initiator. Solid-phase synthesis of DNA initiator was performed by using Mermade-4 DNA synthesizer. The reagents and coupling conditions for standard DNA phosphoramidites were identically adopted for SBiB phosphoramidites, as shown in Table 2. After the DNA initiator synthesis, the CPG-packed column was dried in a vacuum for 15 min. DNA initiators on the dried CPG beads were cleaved and deprotected by treating AMA solution (1:1 mixture of 30% ammonium hydroxide and 40% aqueous methylamine) or 50 mM potassium carbonate for 1.5-4 hours. The deprotected DNA solution was desalted and purified using the Glen-Pak DNA desalting column following the DMT-on desalting procedure provided by the manufacturer (Table 3). To determine DNA initiator concentrations, the absorbance at 260 nm was measured for calculation using the Beer-Lambert law. The corresponding extinction coefficient was calculated by Oligo Analyzer (IDT). The list of DNA and DNA initiators used in this study is demonstrated in Table 4.

TABLE 2
Table for reagents and conditions for solid-
phase DNA and DNA initiator synthesis

| Synthesis step | Reagent | Volume | | Time |
|---|---|---|---|---|
| Wash | Acetonitrile anhydrous | 120 | μL | 3 × 30 s |
| Detritylation | 3% Trichloroacetic acid in DCM | 120 | μL | 2 × 50 s |
| Wash | Acetonitrile anhydrous | 120 | μL | 3 × 30 s |
| Coupling | 0.1M DNA or SBiB | 60 | μL | 4 × 70 s |
| | phosphoramidites | | | |
| | in dry Acetonitrile | | | |
| | 0.25M 5-Ethylthio- | 60 | μL | |
| | 1H-Tetrazole in dry | | | |
| | Acetonitrile | | | |
| Washing | Acetonitrile anhydrous | 120 | μL | 3 × 30 s |
| Capping | 5% Phenoxyacetic | 60 | μL | 2 × 60 s |
| | anhydride in THF/Pyridine | | | |
| | 16% 1-Methylimidazole in THF | 60 | μL | |
| Washing | Acetonitrile anhydrous | 120 | μL | 3 × 30 s |
| Oxidation | 0.02M Iodine in THF/ | 120 | μL | 2 × 50 s |
| | Pyridine/Water | | | |
| Washing | Acetonitrile anhydrous | 120 | μL | 3 × 30 s |
| Deprotection | 1:1 mixture of 30% | 1 | ml | <3 hrs |
| and cleavage | NH₄OH and 40% aqueous | | | |

Methylamine or 50 mM
potassium carbonate

TABLE 3
Table for DMT-on DNA desalting conditions and reagents

| Synthesis step | Reagent | Volume |
|---|---|---|
| Rinse (1) | Acetonitrile | 1 mL |
| Rinse (2) | 2M Triethylamine Acetate buffer | 1 mL |
| Loading | Deprotected DNA in 50 mg/mL NaCl | 2 mL |
| Wash (1) | 5% Acetonitrile in 100 mg/mL NaCl | 2 mL |
| Detritylation | 2% Trifluoroacetic acid in water | 2 mL |
| Wash (2) | Water | 2 mL |
| Elution | 50% Acetonitrile in water | 2 mL |
| | containing 0.5% NH₄OH | |

TABLE 4
Table for the sequences of oligonucleotide
macroinitiators used in studies

| Name of | | Mass, | Mass, |
|---|---|---|---|
| oligonucleotide | Sequence (5′ to 3′) | expected | found |
| T9, unmodified | TTTTTT TTT | 2676.8 | Purchased |
| T4-SBiB-T4 | TTT T /i/ TTTT | 2673.7 | 2671.9 |
| T8-SBiB | /i/ TT TTT TTT | 2673.7 | 2672.8 |
| T8-ibbr | /ibbr/ TTTTTT TT | 2670.7 | 2672.3 |
| T8-s-SB1B5 | TT /i/ T /i/ T /i/ T /i/ T /i/ TT | 3877.1 | 3877.8 |
| T8-t-SBiB5 | /i/ /i/ /i/ /i/ /i/ TTTTTT TT | 3877.1 | 3876.9 |
| T8-m-SB1B5 | TTT T /i/ /i/ /i/ /i/ /i/ TTTT | 3877.1 | 3875.5 |
| Alpha hairpin | CAA GAT GGT GCG AAA AAA CGC ACC | 9234.1 | Purchased |
| | ATC TTG | | |
| Alpha-m- | CAA GAT GGT GCG AAA /i/ AAA CGC ACC | 9535.2 | 9537.7 |
| SBiB1 | ATC TTG | | |
| Alpha-/-SBiB1 | /i/ CAA GAT GGT GCG AAA AAA CGC ACC | 9535.2 | 9538.0 |
| | ATC TTG | | |
| Beta | ACG TCG AGC AGT CAG AGC TGT | 6472.2 | Purchased |
| Beta* | ACA GCT CTG ACT GCT CGA CGT | 6383.2 | Purchased |
| Beta*_{Tmm} | ACA GCT CTG T ACT GCT CGA CGT | 6687.4 | Purchased |
| Beta-SBiB1 | ACG TCG AGC AGT /i/ CAG AGC TGT | 6773.3 | 6771.4 |
| Beta-SBiB2 | ACG TCG AGC /i/ AGT /i/ CAG AGC TGT | 7074.4 | 7076.5 |
| Beta-SBiB3 | /i/ ACG TCG AGC AGT /i/ CAG AGC TG | 7375.5 | 7374.5 |
| | /i/ T | | |
| Beta-SB1B5 | ACG TCG AGC AGT /i/ /i/ /i/ /i/ /i/ | 7977.7 | 7980.1 |
| | CAG AGC TGT | | |
| T12-pc-SB1B3 | /i/ /PC/ /i/ TT /PC/ TT /i/ TTT TTT | 5011.1 | 5013.3 |
| | TT | | |
| T10-SB1B | /i/ T TTT TTT TTT | 3280.7 | 3282.4 |
| U20-SB1B | /i/ rUrU rUrUrU rUrUrU rUrUrU rUrUrU | 6362.4 | 6362.5 |
| | rUrUrU rUrUrU | | |
| U12-pc-SBiB2 | /i/ rUrUrUrUrUrU /PC/ /i/ rUrUrU | 8663.2 | 8661.4 |
| | rUrUrU | | |

i: SBiB phosphoramidite reported in this study
ibbr: ATRP initiator phosphoramidite with 4 carbon spacers
PC:Photo cleavable linker; 3-(4,4′-Dimethoxytrityl)-1-(2-nitrophenyl)-propan-1-yl-[(2-cyanoethyl)-(N,N-diisopropyl)]-phosphoramidite

Preparation of RNA initiator. RNA initiators were synthesized in the Mermade oligonucleotide synthesizer at a 1 μM scale. The same reaction conditions for the coupling of standard RNA phosphoramidites were adopted for the coupling of SBiB phosphoramidite without change. See Table 5. After the solid-phase synthesis, oligonucleotides were cleaved from CPG solid support and bases were by the treatment of AMA solution (1:1 mixture of 30% ammonium hydroxide and 40% aqueous methylamine) or 50 mM potassium carbonate for 1.5-4 hours. The CPG beads were removed by using a Pierce centrifuge filter. The filtered solution was lyophilized overnight and redissolved in DMSO anhydrous. Then, triethylamine trihydrofluoride (TEA·3HF) and triethylamine were introduced and incubated for 2.5 hours at 65° C. Following the incubation, Glen-Pak RNA quenching buffer was added and the fully deprotected RNA was desalted and purified using the Glen-Pak RNA desalting column following the DMT-on desalting procedure provided by the manufacturer. The concentration of RNA was determined by measuring the absorbance at 260 nm followed by calculation using Beer-Lambert law and the corresponding extinction coefficient calculated by IDT.

TABLE 5
Reaction conditions for the solid-phase RNA and RNA
initiator synthesis in oligonucleotide synthesizer.

| Synthesis step | Reagent | Volume | | Time |
|---|---|---|---|---|
| Wash | Acetonitrile anhydrous | 120 | μL | 3 × 30 s |
| Detritylation | 3% Trichloroacetic acid in DCM | 120 | μL | 2 × 50 s |
| Wash | Acetonitrile anhydrous | 120 | μL | 3 × 30 s |
| Coupling | 0.1M RNA or SBiB | 60 | μL | 4 × 70 s |
| | phosphoramidites in dry | | | |
| | Acetonitrile | | | |
| | 0.25M 5-Ethylthio-1H- | 60 | μL | |
| | Tetrazole in dry Acetonitrile | | | |
| Washing | Acetonitrile anhydrous | 120 | μL | 3 × 30 s |
| Capping | 5% Phenoxyacetic | 60 | μL | 2 × 60 s |
| | anhydride in THF/Pyridine | | | |
| | 16% 1-Methylimidazole in THF | 60 | μL | |
| Washing | Acetonitrile anhydrous | 120 | μL | 3 × 30 s |
| Oxidation | 0.02M Iodine in THF/ | 120 | μL | 2 × 50 s |
| | Pyridine/Water | | | |
| Washing | Acetonitrile anhydrous | 120 | μL | 3 × 30 s |
| Deprotection | 1:1 mixture of 30% | 1 | ml | <4 hrs |
| and cleavage | NH₄OH and 40% aqueous | | | |
| | Methylamine or 50 mM | | | |
| | potassium carbonate | | | |
| Lyophilization | N/A | N/A | | 16 hrs |
| 2′ OH | TEA 3HF | 75 | μL | 2.5 hrs |
| deprotection | | | | |
| Quenching | Glen-Pak RNA Quenching Buffer | 1.75 | mL | N/A |

General procedure for grafting from DNA initiators. Prior to polymerization, stock solutions for EY/Cu-mediated ATRP were prepared following the recipe presented below.
CuBr₂ stock: CuBr₂ (15 mg) in 1194 μL of 50:50 DMSO:water
TPMA stock: TPMA (15 mg) in 153.6 μL of DMSO
EY stock: EY (9.72 mg) in 10 ml of DMSO
OEOMA₅₀₀ stock: OEOMA₅₀₀ (250 mg) in 1 ml water

The standard procedure for grafting from the DNA initiator via EY/Cu-mediated ATRP is as follows: 150 μL of monomer stock, 4 μL of CuBr₂ stock, 2 μL of TPMA stock, 2.5 μL of EY stock, 25 μL of 10×PBS and DNA or HEBiB initiator (final concentration of 0.3 mM to 1.5 mM, depending on the target DP) was mixed, and the volume was brought to 250 μL by addition of water. Then the reaction mixture was thoroughly mixed by pipetting and transferred to a 250 μL glass insert in a 0.5-dram glass vial as shown in FIG. S6A. Without deoxygenation, green light (λ=520 nm, 3.7 mW cm) was irradiated to the open reaction vial for polymerization at r.t. for 30 min. Following polymerization, 50 μL of the product was taken for ¹H NMR to measure the monomer conversion, and 200 μL was used for analysis by GPC.

Kinetic study for grafting from DNA initiator. The stock solution for the standard polymerization procedure was used. 450 μL of OEOMA₅₀₀ stock, 12 μL of CuBr₂ stock, 6 μL of TPMA stock, 7.5 μL of EY stock, 75 μL of 10×PBS, and DNA initiator (final concentration of 0.5 mM) were mixed, and the volume was brought to 750 μL by adding water. The reaction mixture was thoroughly mixed by pipetting and transferred to a 1 ml glass shell vial. Without deoxygenation, the polymerization was conducted at r.t. under the irradiation of green light (λ=520 nm, 3.7 mW cm⁻²) for 60 min with an open vial system. For the determination of monomer conversion by ¹H NMR, 50 μL was taken from the polymerization mixture at various time frames (0-60 min). After 60 min of polymerization, 200 μL was taken for analysis by GPC (DMF as eluent).

Steric hindrance study. T4-SBiB-T4 and T8-SBiB were synthesized and desalted following the standard DMT-on desalting protocol. T8-ibbr was prepared following the previously reported procedure [ref]. Standard polymerization conditions were adopted at the target DP of 400 in a 250 μL reaction scale (0.75 mM of final DNA initiator concentration). After 30 min of polymerization, 50 μL of the product was taken for ¹H NMR analysis to measure the monomer conversion, and 200 μL was used for analysis by GPC (DMF as eluent).

Grafting methyl acrylate (MA) from DNA initiator on solid support. Prior to polymerization, 8 M MA stock (3444 mg in DMSO, final volume of 5 mL) and 300 mM ethyl α-bromoisobutyrate (EBiB) stock (115 mg in DMSO, final volume of 2 mL) were prepared. T8-SBiB DNA initiator was synthesized at the 1 μmole scale using the CPG solid support with the pore size of 1000 Å, following the procedure presented above. Next, 962.5 μL of MA stock, 68.5 μL of CuBr₂ stock, 68.7 μL of Me₆TREN stock, 7.75 μL, of EYH₂ stock, 70 μL of DMF, 116.7 μL of EBiB stock, 175.9 μL of DMSO, and the CPG beads with the 1 μmoles of DNA initiator (ca. 25 mg) was introduced into the 0.5-dram vial. [MA]/[EBiB]/[CuBr₂][Me₆TREN]/[EYH₂]=220/1/0.11/0.66/0.000332, [MA]=5500 mM. After 45 min of polymerization with stirring under the green light irradiation (λ=520 nm, 3.7 mW cm⁻²), the solid support was filtered and thoroughly washed with THF and acetonitrile. Next, 500 μL of NH₄OH was mixed with the CPG support to cleave DNA-poly(methyl acrylate) (DNA-pMA) block copolymer from the solid support. After 2 hours of incubation at room temperature, the supernatant with DNA-pMA was collected and characterized by DMF GPC. Monomer conversion was determined by 1H NMR spectroscopy using DMF as the internal standard.

Double-stranded DNA preparation and melting curve analysis. Desired single-stranded DNA (Beta or Beta-SBiB) was mixed with a stoichiometric amount of complementary DNA (Beta* or Beta*_{Tmm}) at a final concentration of 20 μM in 1×PBS. The DNA mixture was placed in a heat block at 95° C. for 5 min. Following the heating process, the heat block was turned off and slowly cooled to room temperature (approx. 0.5° C./min). The hybridization efficiency of Beta and Beta-SBiB1 with Beta* or Beta*_{Tmm} was confirmed by 15% non-denaturing PAGE. For the visualization of DNA, GelGreen dye was used for DNA staining.

For the melting curve analysis shown in FIG. S16, Beta DNA with a polymer modification was synthesized by grafting from Beta-SBiB1 initiator and the resulting Beta-pOEOMA was purified by using the MWCO filter followed by hybridization with complementary DNA (i.e., Beta*) following the procedure described above. Next, the Beta DNA duplexes with or without the single SBiB or polymer modification were prepared as demonstrated above. The dsDNA (final concentration of 2.5 μM) was mixed with EvaGreen (final concentration of 1×), 1×PBS and NaCl (final concentration of 80 mM) at a final volume of 50 μl. In a real-time PCR thermal cycler, the mixture was incubated at 25° C. for 5 min followed by gradual heating to 95° C. at a rate of 0.9° C./min while recording the fluorescence intensity of EvaGreen.

For the melting curve analysis shown in FIG. 4, the desired DNA polymer conjugates were synthesized by grafting from the appropriate DNA initiator under the standard polymerization condition for 30 min. The DNA polymer conjugates were purified by using the MWCO filter and, if necessary, hybridized with Beta* following the previously mentioned protocol. Next the DNA structures (final concentration of 20 μM) with or without initiator or polymer modification were mixed with EvaGreen (final concentration of 1.5×), 1×PBS, NaCl (final concentration of 80 mM), MgCl₂ (final concentration of 20 mM). In a real-time PCR thermal cycler, the mixture was incubated at 25° C. for 5 min followed by gradual heating to 95° C. at a rate of 0.9° C./min while recording the fluorescence intensity of EvaGreen.

Grafting from dsDNA initiator. 2× concentrated OEOMA₅₀₀ stock solution was prepared by dissolving 500 mg of OEMA₅₀₀ in water at a final volume of 1 ml. dsDNA initiator stock was prepared by mixing Beta-SBiB1 (final concentration of 0.833 mM) with Beta* (1.1 equiv., 0.916 mM) in 1×PBS at a final volume of 150 μL followed by temperature annealing. The DNA mixture was heated to 95° C. for 5 min on a heat block, and the heat block was turned off to slowly cool the DNA mixture to room temperature. For the polymerization from the dsDNA initiator, 75 μL of 2×OEOMA₅₀₀ stock, 4 μL of CuBr₂ stock, 2 μL of TPMA stock, 2.5 μL of EY stock, and 25 μL of 10×PBS were added to dsDNA initiator stock. The reaction mixture was thoroughly mixed by pipetting and transferred to a 250 μL glass insert in a 0.5-dram glass vial. Without deoxygenation, green light (λ=520 nm, 3.7 mW cm²) was irradiated to the open reaction vial for polymerization at r.t. for 30 min. Following polymerization, 50 μL of the product was taken for ¹H NMR to measure the monomer conversion, and 200 μL was used to determine apparent Mₙ and Ð by aqueous GPC (calibrated to PEO).

Grafting from the initiator with PC linkage and SEC-MALS analysis. As an example, polymerization and subsequent UV irradiation process for DNA initiator are demonstrated. The T12-pc-SBiB3 initiator was synthesized and desalted by standard DMT-on desalting protocol. Standard polymerization condition was adopted at the target DP of 800 in 250 μL reaction scale (0.375 mM of final DNA initiator concentration). After 30 min of polymerization, 50 μL of the product was taken for ¹H NMR analysis to measure the monomer conversion. 200 μL was taken and washed 6 times with water using a 100K Amicon ultra centrifugal filter. The washed sample was further purified by passing over the Sep-Pak C18 cartridge to remove EY. The solvent was evaporated, and DNA-polymer was redissolved in 1×DBPS followed by analysis using SEC-MALS to obtain absolute molecular weight and Ð. For the cleavage of polymer tethers from the DNA backbone, UV light (λ=365 nm, 6.0 mW cm²) was irradiated for 5 min. After UV irradiation, the mixture was analyzed by SEC-MALS to obtain absolute molecular weight and Ð of cleaved polymer tethers.

Stability assay in 10% FBS. Beta-p(OEOMA)3 was prepared by grafting from Beta-SBiB3 (final concentration of 0.75 mM) under the standard polymerization conditions at the target DP of 400. Followed by polymerization, 50 μL of the product was taken for ¹H NMR analysis to measure the monomer conversion. 200 μL was taken and washed 6 times with water using a 100K Amicon ultra centrifugal filter. The washed polymer was further purified by passing through the Sep-Pak C18 cartridge. The solvent was evaporated, and Beta-p(OEOMA)3 was redissolved in water. Purified Beta-p(OEOMA)3 was analyzed by SEC-MALS to obtain absolute molecular weight and Ð.

For the stability assay in 10% FBS, Beta-p(OEOMA)3, Beta-SBiB3 or Beta (final concentration of 5.3 μM) was mixed with FBS (final concentration of 10%) followed by incubation at 37° C. for 0-24 hours. For the control experiment of incubation of unmodified Beta DNA in the presence of free polymer, pOEOMA (M_{n,MALS}=169 000) was synthesized under the standard polymerization condition using HEBiB as the initiator, purified following the previously described procedure, and incubated with Beta DNA at the final concentration of 2.5 mg/mL (ca. 15 μM). After incubation, the solutions were heated at 95° C. for 10 min and slowly cooled to room temperature. The amount of remaining DNA was examined by 5% non-denaturing PAGE carried out at 120 V for 50 min. Following gel electrophoresis, the gels were stained by 3× GelGreen in 0.5×TBE for 10 min and analyzed by Typhoon FLA 9000 image scanner. ImageQuant TL software was used for quantification. The experiment was repeated, and standard deviations were calculated from three different batches.

For the stability assay under the specific nuclease treatment, Beta-p(OEOMA)3 or Beta (final concentration of 8 μM) was mixed with DNAse I (33 U/ml) or Exonuclease VII (660 U/ml) and reaction buffer (for DNAse I, 10 mM Tris-HCl. 2.5 mM MgCl₂, 0.5 mM CaCl₂; for Exonuclease VII, 50 mM Tris-HCl, 50 mM sodium phosphate, 8 mM EDTA, 10 mM 2-mercaptoethanol) followed by incubation at 37° C. for 15 min (DNAse I) or 30 min (Exonuclease VII), respectively. After incubation, the solutions were heated at 95° C. for 10 min and slowly cooled to room temperature. The amount of remaining DNA was examined by 15% non-denaturing PAGE carried out at 110 V for 30 min. Following gel electrophoresis, the gels were stained by 3× GelGreen in 0.5×TBE for 10 min and analyzed by Typhoon FLA 9000 image scanner. ImageQuant TL software was used for quantification.

Examination of stability of SBiB in the 2′-OH in RNA deprotection condition (at 65° C. for 2.5 h in TEA·3HF dissolved in DMSO). The T10-SBiB DNA initiator was synthesized following the standard DNA synthesis and DMT-on deprotection condition. 500 nmoles of the T10-SBiB DNA initiator were taken and dissolved in 115 μL of 50% DMSO in water. The DNA initiator was treated with 60 μL of TEA and 75 μL of TEA·3HF and gently mixed followed by incubation at 65° C. for 2.5 hr. Next, the reaction was quenched by adding Glen-Pak RNA Quenching Buffer to the deprotection solution. Following the quenching step, The T10-SBiB DNA initiator was desalted by using the Glen-Pak RNA desalting column following the procedure suggested by the manufacturer. The T10-SBiB DNA initiator with or without TEA·3HF treatment was used for polymerization under the general polymerization condition at the target DP of 300 ([T10-SBiB]=1 mM) followed by analysis by DMF GPC.

Kinetic study of grafting OEOMA₅₀₀ from RNA. For the kinetic study of polymerization from the RNA initiator, the reaction mixture was prepared as follows: 12 μL of CuBr₂ stock, 6 μL of TPMA stock, 450 μL of OEOMA₅₀₀ stock, 7.5 μL of EY stock, 75 μL of 10×PBS and initiator (final concentration of 0.5 mM) was mixed, and the final volume was brought to 750 μL by adding water. The reaction mixture was thoroughly mixed and transferred into a 1 mL flat-bottomed glass insert. Then, green LED light (λ=520 nm, 7.6 mW cm⁻²) was irradiated to the reaction mixture at r.t. for 60 min. For the determination of monomer conversion, 50 μL of the mixture was taken at certain time frames (0-60 min) and analyzed by ¹H NMR. After 60 min of reaction, the reaction was stopped by turning the LED light off and blowing air into the reaction mixture. 200 μL of the polymerization product was taken and purified by using a molecular weight cut-off filter or precipitation for GPC analysis.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A method of synthesizing a polynucleotide composition, comprising:
performing a synthesis of the polynucleotide composition via phosphoramidite chemistry, solid-phase supported synthesis, to create a polynucleotide chain;
during at least one cycle of the synthesis, coupling a reversible-deactivation-radical-polymerization-(an RDRP)-phophoramidite reagent in a growing chain of the polynucleotide composition, the RDRP-phophoramidite reagent comprising a phosphoramidite compound conjugated to an RDRP initiator or a chain transfer agent for RDRP via a moiety comprising a protected hydroxyl group, wherein a hydroxyl protecting group of the protected hydroxyl group is stable to polynucleotide synthesis conditions.

2. The method of claim 1 further comprising
after coupling the RDRP-phophoramidite reagent, removing the hydroxyl protecting group therefrom to form a hydroxyl group, and
coupling one of a phosphoramidite nucleoside or another RDRP-phosphoramidite reagent via the hydroxyl group.

3. The method of claim 1 further comprising coupling an RDRP-phophoramidite reagent in more than one cycle of the synthesis of the polynucleotide composition.

4. The method of claim 2 wherein the phosphoramidite nucleoside is a phosphoramidite deoxyribonucleoside or a phosphoramidite ribonucleoside.

5. (canceled)

6. The method of claim 2 wherein the hydroxyl protecting group is a dimethoxytrityl group or a monomethoxytrityl group.

7. The method of claim 1 wherein the RDRP-phophoramidite reagent has the formula: 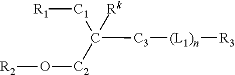
wherein R₁ is a phosphoramidityl group, R₂ is the hydroxyl protecting group, C₁, C₂, and C₃ are independently a base-stable spacer group, R^{k} is selected from the group of H and C1-C6 alkyl, C₃ may be present or absent, L₁ is a base-stable linking group, wherein n is an integer in the range of 0 to 40, and R₃ is the RDRP initiator or the chain transfer agent for RDRP.

8. The method of claim 7 wherein C₁, C₂, and C₃ are independently selected from the group consisting of: 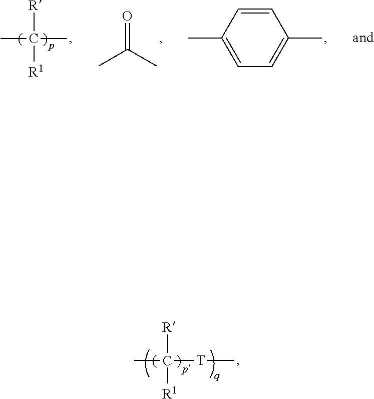
wherein T is selected from the group O, S, —C(O)NH— or —NHC(O)—, p is an integer in the range of 0 to 18, R^{k} and R^{l} are independently selected from the group consisting of H and C1-C6 alkyl, p′ is an integer in the range of 0 to 18 and q is an integer in the range of 1 to 18.

9. The method of claim 7 wherein L₁ is selected from the group consisting of: 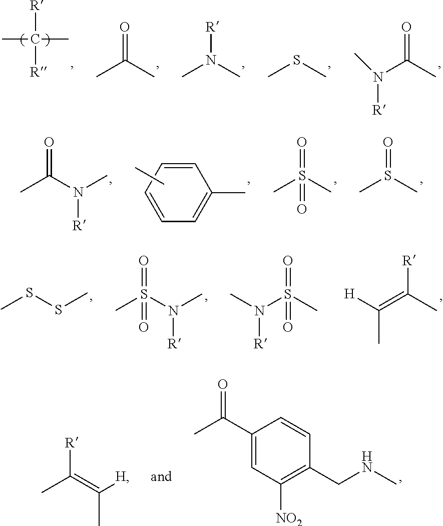
wherein R′ and R″ are independently selected from the group consisting of H, alkyl and aryl.

10. The method of claim 7 wherein the phosphoramidityl group has the formula: 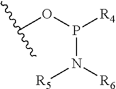
wherein R₄ is a phosphate protecting group, and R₅ and R₆ are independently selected from the group consisting of a C1-C12 alkyl group, a C1-C10 branched alkyl group, and a C3-C8 cyclic alkyl group.

11. The method of claim 7 wherein R₂ is a dimethoxytrityl group or a monomethoxytrityl group.

12. The method of claim 10 wherein R₄ is —O—(CH₂)ₙCN, wherein n is an integer in the range of 1 to 5.

13. The method of claim 7 wherein R₃ has the formula: 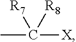
wherein X is a homolytically cleavable group or a group activated by degenerative radical exchange;
R₇ and R₈ are each independently selected from the group consisting of a homolytically cleavable group, a group activated by degenerative radical exchange, H, C1-C20 alkyl, C3-C8 cycloalkyl, C(═Y)R₉, C(═Y)NR₁₀R₁₁, COCl, OH, CN, C2-C20 alkenyl, C2-C20 alkynyl, oxiranyl, glycidyl, aryl, heterocyclyl, aralkyl, aralkenyl, C1-C6 alkyl in which from 1 to all of the hydrogen atoms are replaced with halogen and C1-C6 alkyl substituted with from 1 to 3 substituents selected from the group consisting of C1-C4 alkoxy, aryl, heterocyclyl, C(═Y)R₉, C(═Y)NR₁₀R₁₁, oxiranyl and glycidyl,
wherein R₉ is C1-C20 alkyl, C1-C20 alkoxy, aryloxy or heterocyclyloxy, and R₁₀ and R₁₁ are independently H, or C1-C20 alkyl, or R₁₀ and R₁₁ may be joined together to form an alkylene group of from 2 to 5 carbon atoms, wherein Y is NR₁₂ or O and R₁₂ is H, straight or branched C1-C20 alkyl or aryl.

14. The method of claim 13 wherein X is selected from the group consisting of Cl, Br, I, nitroxyl, organotellurium, organostibine, organobismuthine, and —S—C(═S)—Z, wherein Z is selected from the group consisting of alkyl, alkoxy, alkylthio, aryl, and heteroaryl.

15.-19. (canceled)

20. The method of claim 7 wherein C₁ is —CH₂—, and C₂ is —CH₂— or —CHCH₃— and C₃ is 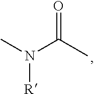
wherein R′ is H and n is 0.

21.-23. (canceled)

24. The method of claim 13 wherein X is Br, R₇ is methyl and R₈ is methyl.

25. The method of claim 1, further comprising growing a polymer from the RDRP initiator or from a site of the chain transfer agent for RDRP via an RDRP polymerization.

26. The method of claim 25 wherein the RDRP initiator is an ATRP-initiator or the chain transfer agent for RDRP is a chain transfer agent for RAFT.

27. The method of claim 25 wherein the polymer is grown from the RDRP initiator or from the site of the chain transfer agent for RDRP while the polynucleotide composition is attached to a solid support used in the solid-phase supported synthesis or the polymer is grown from the RDRP initiator after the polynucleotide chain is detached from a solid support used in the solid-phase supported synthesis.

28.-30. (canceled)

31. A composition comprising a phosphoramidite compound conjugated to an RDRP initiator or a chain transfer agent for RDRP via a moiety comprising a protected hydroxyl group, wherein a hydroxyl protecting group of the protected hydroxyl group is stable to polynucleotide synthesis conditions.

32. The composition of claim 31 wherein the hydroxyl protecting group is a dimethoxytrityl group or a monomethoxytrityl group.

33. The composition of claim 31 having the formula: 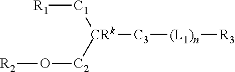
wherein R₁ is a phosphoramidityl group, R₂ is the hydroxyl protecting group, R^{k} is selected from the group consisting of H and C1-C6 alkyl, C₁, C₂, and C₃ are independently a base-stable spacer group, C3 may be present or absent, L₁ is a base-stable linking group, n is an integer in the range of 0 to 40, and R₃ is the RDRP initiator or the chain transfer agent for RDRP.

34. The composition of claim 33 wherein C₁, C₂, and C₃ are independently selected from the group consisting of: 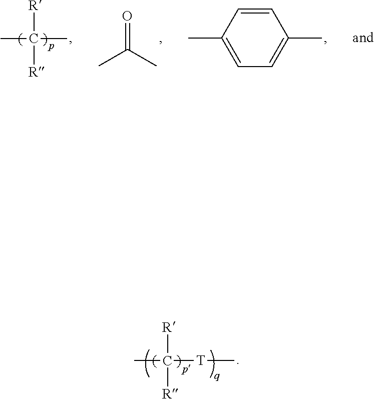
wherein T is selected from the group O, S, —C(O)NH— or —NHC(O)—, R′ and R″ are independently selected from the group consisting of H, alky, and aryl, p is an integer in the range of 0 and 18, p′ is an integer between 0 and 18, and q is an integer in the range of 1 to 18.

35. The composition of claim 33 wherein L₁ is selected from the group consisting of: 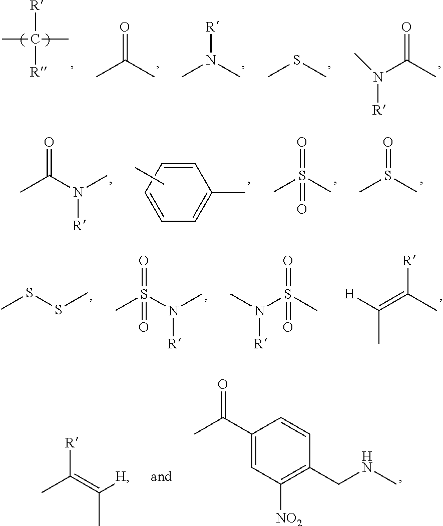
wherein R′ and R″ are independently selected from the group consisting of H, alkyl and aryl.

36. The composition of claim 33 wherein the phosphoramidityl group has the formula: 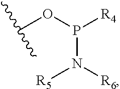
wherein R4 is a phosphate protecting group, and R₅ and R₆ are independently selected from the group consisting of a C1-C12 alkyl group, C₁-C10 branched alkyl group, and a C3-C8 cyclic alkyl group.

37. The composition of claim 33 wherein R₂ is a dimethoxytrityl group or a monomethoxytrityl group.

38. The composition of claim 36 wherein R₄ is —O—(CH₂)ₙCN, wherein n is an integer in the range of 1 to 5.

39. The composition of claim 33 wherein R₃ has the formula: 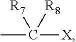
wherein X is a homolytically cleavable group or a group activated by degenerative radical exchange;
R₇ and R₈ are each independently selected from the group consisting of a homolytically cleavable group, a group activated by degenerative radical exchange, H, C1-C20 alkyl, C3-C8 cycloalkyl, C(═Y)R₉, C(═Y)NR₁₀R₁₁, COCl, OH, CN, C2-C20 alkenyl, C2-C20 alkynyl, oxiranyl, glycidyl, aryl, heterocyclyl, aralkyl, aralkenyl, C1-C6 alkyl in which from 1 to all of the hydrogen atoms are replaced with halogen and C1-C6 alkyl substituted with from 1 to 3 substituents selected from the group consisting of C1-C4 alkoxy, aryl, heterocyclyl, C(═Y)R₉, C(═Y)NR₁₀R₁₁, oxiranyl and glycidyl,
wherein R₉ is C1-C20 alkyl, C1-C20 alkoxy, aryloxy or heterocyclyloxy, and R₁₀ and R₁₁ are independently H, or C1-C20 alkyl, or R₁₀ and R₁₁ may be joined together to form an alkylene group of from 2 to 5 carbon atoms, wherein Y is NR₁₂ or O and R₁₂ is H, straight or branched C1-C20 alkyl or aryl.

40.-45. (canceled)
